(19) United States
(12) Patent Application Publication
(10) Pub. No.: US 2022/0099637 A1
(43) Pub. Date: Mar. 31, 2022

(54) METHODS OF ANALYSIS USING IN-SAMPLE CALIBRATION CURVE BY MULTIPLE ISOTOPOLOGUE REACTION MONITORING

(71) Applicant: Bristol-Myers Squibb Company, Princeton, NJ (US)

(72) Inventors: Huidong GU, Princeton, NJ (US); Yue ZHAO, Princeton, NJ (US); Jianing ZENG, Princeton, NJ (US)

(73) Assignee: Bristol-Myers Squibb Company, Princeton, NJ (US)

(21) Appl. No.: 17/299,699

(22) PCT Filed: Dec. 3, 2019
(86) PCT No.: PCT/US2019/064299

§ 371 Date: Jun. 3, 2021

Related U.S. Application Data
(60) Provisional application No. 62/775,318, filed on Dec. 4, 2018.

(51) Int. Cl. G01N 30/72; G01N 1/40; G01N 33/68

(52) U.S. Cl. CPC G01N 30/7233; G01N 1/4055; G01N 2030/027; G01N 33/6848; G01N 2458/15; G01N 33/6854

(57) ABSTRACT

This disclosure provides several methods in LC-MS/MS analysis: (1) a method of LC-MS/MS analysis technique to determine the analyte concentration of a sample wherein an In-Sample Calibration Curve (ISCC) is used instead of an external calibration curve through monitoring of multiple isotopologue transitions of an added stable isotopically labeled (SIL) analyte in each sample via MS/MS in multiple isotopologue reaction monitoring (MIRM) mode; (2) a method of LC-MS/MS analysis to determine the analyte concentration of a sample wherein a One-Sample Multipoint External Calibration Curve (OSMECC) is used instead of a multisample external calibration curve; and (3) a method of LC-MS/MS analysis to determine the analyte concentration of a sample with an analyte concentration beyond the assay's ULOQ wherein isotope sample dilution is used instead of diluting sample physically during sample preparation based on calculating the isotopic abundance of the MIRM channel monitored.

### CROSS-REFERENCE TO RELATED APPLICATIONS

This application claims priority to U.S. Provisional Application No. 62/775,318, filed Dec. 4, 2018. The entire contents of U.S. Provisional Application No. 62/775,318 are hereby incorporated herein by reference.

### REFERENCE TO A SEQUENCE LISTING SUBMITTED ELECTRONICALLY VIA EFS-WEB

This application includes a Sequence Listing submitted electronically via EFS-Web (name: “3338_148PC01_SL_ST25.txt”; size: 844 bytes; and created on: Dec. 3, 2019), which is hereby incorporated by reference in its entirety.

### BACKGROUND OF THE DISCLOSURE

With the recent rapid development in translational medicine research, quantitative determination of biomarkers in pre-clinical and clinical studies, and quantitative proteomics have been playing even more important roles in molecular and cellular biology research, drug discovery and development, such as identifying and validating potential biomarkers, facilitating patient stratification and dose selection, serving as surrogate endpoints and establishing PK/PD relationship at the site of action. It is well known that the use of external calibration curves prepared in the same biological matrix as the incurred study samples, and the use of a stable isotopically labeled (SIL) analyte in both of the external calibration curves and incurred study samples as an assay internal standard are the keys in developing accurate and robust liquid chromatography-tandem mass spectrometry (LC-MS/MS) assays for the quantitative determination of small molecule drugs and biotherapeutics in biological matrices.

However, for biomarker measurement in pre-clinical and clinical studies, due to a biomarkers' endogenous nature, the use of external calibration curves with authentic reference standards in authentic matrix is not possible in many cases. Preparing an external calibration curve with a SIL surrogate analyte in authentic matrix is one of the alternatives and can provide the same assay performances as those of an assay using an authentic analyte in authentic matrix since the SIL surrogate analyte and the authentic analyte have the “identical” physicochemical properties regarding sample extraction, chromatographic separation, MS ionization, fragmentation and detection. However, this approach is not easily available as another version of the SIL analyte is needed as the assay internal standard, and access to two versions of the SIL analyte is very costly and time consuming, especially for the analysis of proteins. Therefore, there is a need to develop other approaches for the quantitative LC-MS/MS analysis of biomarkers, drugs, or their metabolites in preclinical or clinical stages of drug development.

### SUMMARY OF THE DISCLOSURE

The present disclosure is related to an LC-MS/MS technique for quantifying the concentration of at least one analyte in a sample, the method comprising adding one or more known amount(s) of stable isotopically labeled (SIL) analyte(s) to a sample containing at least one analyte to construct one or more In-Sample Calibration Curve(s) (ISCC) by Multiple Isotopologue Reaction Monitoring (MIRM) of each added SIL analyte(s), wherein the MIRM of an SIL analyte refers to multiple reaction monitoring of multiple isotope transitions of the SIL analyte; wherein the ISCC for each analyte is constructed in the sample based on the relationship between the calculated theoretical isotopic abundances (analyte concentration equivalents) in the MIRM transitions and the measured tandem mass spectrometry (MS/MS) peak areas in the corresponding MIRM transitions; wherein the concentration of the at least one analyte in the sample is quantified using the established ISCC and the measured peak area for the analyte from a liquid chromatography-tandem mass spectrometry (LC-MS/MS) process, and wherein a tandem mass spectrometer is operated in multiple reaction monitoring mode.

In some aspects, the analyte, the SIL analyte, and the naturally occurring isotopologues of the SIL analyte are ionized in the mass spectrometer to produce protonated (or deprotonated) parent ions of the analyte, the SIL analyte and the naturally occurring isotopologues of the SIL analyte. In some aspects, the parent ions of the analyte, the parent ions of the SIL analyte, and the parent ions of the naturally occurring isotopologues of the SIL analyte in the mass spectrometer are fragmented at the same cleavage site to produce neutral losses and daughter ions;

In some aspects, the transition from the parent ion to the daughter ion for the analyte is monitored in the mass spectrometer and a peak area for the transition from the parent ion to the daughter ion for the analyte is measured. In some aspects, the selected multiple transitions from parent ions of the SIL analyte and the parent ions of the naturally occurring isotopologues of the SIL analyte to the daughter ions of the SIL analyte and the daughter ions of the naturally occurring isotopologues of the SIL analyte are monitored in the mass spectrometer (“multiple isotopologue reaction monitoring” or “MIRM”);

In some aspects, a peak area of each of the MIRM transitions is measured, wherein the MIRM transitions comprise the selected transitions from parent ions of the SIL analyte and the parent ions of the naturally occurring isotopologues of the SIL analyte to the daughter ions of the SIL analyte and the daughter ions of the naturally occurring isotopologues of the SIL analyte.

In some aspects, an In-Sample Calibration Curve is generated based on the relationship between the measured peak areas in the MIRM transitions of the SIL analyte and the naturally occurring isotopologues of the SIL analyte, and the analyte concentration equivalents for each of the MIRM transitions.

In some aspects, the analyte concentration equivalent for each MIRM transition is calculated from a theoretical isotopic abundance of the corresponding MIRM transition of the SIL analyte or the naturally occurring isotopologues of the SIL analyte, wherein the theoretical isotopic abundance is calculated using a methodology published in Analytical Chemistry, 2012, 84(11), 4844-4850, wherein the methodology is calculated based on the isotope distributions of the neutral loss and the daughter ion of the SIL analyte. In other aspects, for a unit resolution triple quadrupole mass spectrometer, the theoretical isotopic abundance for each of the MIRM transition (m/z) from (p+Zₚ+α)/Zₚ to (d+Z_{d}+β)/Z_{d} of the SIL analyte and the naturally occurring isotopologues of the SIL analyte is calculated based on formula (I):

Isotopic abundance in an MIRM transition of (p+Zₚ+α)/Zₚ→(d+Z_{d}+β)/Z_{d}=[relative isotope distribution of the daughter ion at mass of (d+Z_{d}+β)]*[relative isotope distribution of the neutral loss at mass of n+(α−β)] (I)

Wherein m z is the mass to charge ratio
p is the monoisotopic mass of the parent molecule of the SIL analyte
Zₚ is the number of charge for the parent ion
d is the monoisotopic mass of the daughter fragment of the SIL analyte
Z_{d} is the number of charge for the daughter ion
n is the monoisotopic mass of the neutral loss of the SIL analyte
p=d+n
α and β are integer, they are the number of additional neutrons on the parent ion and daughter ion, respectively, α≥0, β≥0 and α≥β
Zₚ and Z_{d} are integers

In some aspects, the isotopic abundance calculator is at worldwideweb.sisweb.com/mstools/isotope.html (accessed Nov. 10, 2019). In some aspects, wherein the highest analyte concentration equivalent (“Upper Limit of Quantification” or “ULOQ” of the ISCC) is calculated based on formula (II):

(M/V)*(M_{analyte}/M_{SIL analyte}) ng/mL (II)

Wherein M (ng) is the total amount of the SIL analyte added into the sample;
V is the sample volume (mL) before the SIL analyte is added;
M_{analyte} is the molecular weight of the analyte;
M_{SIL analyte} is the molecular weight of the SIL analyte.

In some aspects, one or more of the other analyte concentration equivalents in the MIRM transitions are calculated based on formula (III):

Iₐ*ULOQ (ng/ml) (III)
wherein Iₐ is the calculated theoretical isotopic abundance of a MIRM transition of the SIL analyte or the naturally occurring isotopologues of the SIL analyte.

The present methods are effective to detect or quantify an analyte that is a protein using a corresponding SIL analyte. In some aspects, the analyte is a protein or a peptide. In some aspects, the SIL analyte is a stable isotopically labeled protein or peptide. In some aspects, a parent ion of the analyte and a parent of the SIL analyte comprise at least about 3 amino acids, at least about 4 amino acids, at least about 5 amino acids, at least about 6 amino acids, at least about 7 amino acids, at least about 8 amino acids, at least about 9 amino acids, at least about 10 amino acids, at least about 11 amino acids, at least about 12 amino acids, at least about 13 amino acids, at least about 14 amino acids, at least about 15 amino acids, at least about 16 amino acids, at least about 17 amino acids, at least about 18 amino acids, at least about 19 amino acids, or at least about 20 amino acids.

In some aspects, a parent ion of the analyte and a parent ion of the SIL analyte comprises an amino acid sequence between 4 and 20 amino acids, between 4 and 15 amino acids, between 5 and 15 amino acids, between 4 and 14 amino acids, between 5 and 14 amino acids, between 5 and 13 amino acids, between 5 and 12 amino acids, between 6 and 15 amino acids, between 6 and 14 amino acids, between 6 and 13 amino acids, between 6 and 12 amino acids, between 6 and 11 amino acids, between 6 and 10 amino acids, between 6 and 9 amino acids, between 6 and 8 amino acids, between 7 and 15 amino acids, between 7 and 14 amino acids, between 7 and 13 amino acids, between 7 and 12 amino acids, between 7 and 11 amino acids, between 7 and 10 amino acids, or between 7 and 9 amino acids. In some aspects, the analyte is an antibody. In other aspects, the analyte is a fusion protein. In some aspects, the analyte is a fusion protein comprising a protein and a heterologous moiety. In other aspects, the analyte is an Fc fusion protein. In some aspects, the analyte is PD-1, PD-L1, CD73, an anti-PD-1 antibody, an anti-PD-L1 antibody, an anti-CD73 antibody, or any combination thereof.

The present methods are also effective to detect or quantify an analyte that is a small molecule. In some aspects, the analyte is a small molecule. In some aspects, the SIL analyte is a stable isotopically labeled small molecule. In some aspects, the small molecule has a molar mass of at least about 100 g/mol, at least about 200 g/mol, at least about 300 g/mol, at least about 400 g/mol, at least about 500 g/mol, at least about 600 g/mol, at least about 700 g/mol, at least about 800 g/mol, at least about 900 g/mol, at least about 1000 g/mol, at least about 1100 g/mol, at least about 1200 g/mol, at least about 1300 g/mol, at least about 1400 g/mol, at least about 1500 g/mol, at least about 1600 g/mol, at least about 1700 g/mol, at least about 1800 g/mol, at least about 1900 g/mol, or at least about 2000 g/mol.

The present methods involve the use of a SIL analyte that is labeled with isotopes. In some aspects, the SIL analyte contains at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, or at least about 20 stable isotope labels.

In some aspects, the SIL analyte contains from about 3 to about 20 isotope labels, from about 3 to about 19 isotope labels, from about 3 to about 15 isotope labels, from about 3 to about 10 isotope labels, from about 3 to about 8 isotope labels, from about 3 to about 7 isotope labels, from about 3 to about 6 isotope labels, from about 4 to about 15 isotope labels, from about 4 to about 10 isotope labels, from about 4 to about 8 isotope labels, from about 4 to about 7 isotope labels, from about 4 to about 6 isotope labels, from about 5 to about 8 isotope labels, from about 5 to about 7 isotope labels, from about 6 to about 10 isotope labels, from about 6 to about 8 isotope labels, from about 7 to about 16 isotope labels, from about 7 to about 16 isotope labels, from about 8 to about 16 isotope labels, from about 8 to about 15 isotope labels, from about 9 to about 15 isotope labels, from about 9 to about 14 isotope labels, from about 10 to about 14 isotope labels, from about 10 to about 13 isotope labels, or from about 11 to about 13 isotope labels.

Selection of the MIRM transitions for measurement is an important element of the present methods to ensure a robust and accurate assay performance. In some aspects, each of the measured relative peak area in MIRM transitions of the SIL analyte has less than 15% deviation from the calculated theoretical isotopic abundance in the corresponding MIRM transition of the SIL analyte or the naturally occurring isotopologues of the SIL analyte.

In some aspects, at least one of the measured relative peak area in MIRM transitions has less than 14%, less than 13%, less than 12%, less than 11%, less than 10%, less than 9%, less than 8%, less than 7%, less than 6%, less than 5%, less than 4%, less than 3%, less than 2%, less than 1%, less than 0.1%, less than 0.01%, less than 0.001%, or less than 0.0001%, deviation from the calculated theoretical isotopic abundance in the corresponding MIRM transition of the SIL analyte or the naturally occurring isotopologues of the SIL analyte.

In some aspects, the number of the MIRM transitions is at least two, at least three, at least four, at least five, at least six, at least seven, at least 8, at least 9, at least 10, at least 11, at least 12, at least 13, at least 14, at least 15, at least 16, at least 17, at least 18, at least 19, or at least 20. In some aspects, the number of the MIRM transitions is between 4 and 15.

In some aspects, the analyte concentration equivalents of the highest MIRM transition and the lowest MIRM transition of the SIL analyte is at least about 10, at least about 100, at least about 200, at least about 300, at least about 400, at least about 500, at least about 600, at least about 700, at least about 800, at least about 900, at least about 1000, at least about 1100, at least about 1200, at least about 1300, at least about 1400, at least about 1500, at least about 1600, at least about 1700, at least about 1800, at least about 1900, or at least about 2000 fold difference.

In some aspects, the calculated theoretical isotopic abundance of two selected MIRM transitions at least 0.01% apart, at least 0.05% apart, at least 0.1% apart, at least 0.5% apart, are at least 1% apart, at least 1.5% apart, at least 2% apart, at least 2.5% apart, at least 3% apart, at least 3.5% apart, at least 4% apart, at least 4.5% apart, at least 5% apart, at least 5.5% apart, at least 6% apart, at least 6.5% apart, at least 7% apart, at least 7.5% apart, at least 8% apart, at least 8.5% apart, at least 9% apart, at least 9.5% apart, at least 10% apart, at least 20% apart, at least 30% apart, at least 40% apart, or at least about 50% apart.

In some aspects, the SIL analyte contains less than 5%, less than 4%, less than 3%, less than 2%, less than 1%, less than 0.5%, less than 0.4%, less than 0.3%, less than 0.2%, or less than 0.1% non-labeled analyte.

In some aspects, the label is ²H, ¹³C, ¹⁵N, ³³S, ³⁴S, ³⁶S, ¹⁷O, or ¹⁸O.

The present methods involve mass spectrometry wherein an ion source is used to ionize an analyte (molecule). In some aspects, the one or more protonated or deprotonated molecular(s) are singly charged, doubly charged, triply charged or higher. In some aspects, the mass spectrometer is a triple quadrupole mass spectrometer comprising Q1, Q2 and Q3. In some aspects, the resolutions used for Q1 and Q3 are unit resolution. In other aspects, the resolutions used for Q1 and Q3 are higher than unit resolution. In other aspects, the resolutions used for Q1 and Q3 are different. In other aspects, the resolution used for Q1 is higher than the unit resolution of Q3.

In some aspects, an In-Sample Calibration Curve (ISCC) composition (i.e. a known amount of the SIL-analyte) is added before or during the sample preparation.

The present methods are highly effective and improving the total required instrument time especially in the instance where an external calibration curve does not have to be run on the instrument. In some aspects, the method reduces a total instrument run time. In some aspects, an external calibration curve is not used. In some aspects, the analyte is a biomarker. In some aspects, the analyte is a metabolite. In some aspects, the analyte is a small molecule drug. In some aspects, the analyte is a peptide. In some aspects, the analyte is a protein.

The present methods are effective for detecting or quantifying analytes from a variety of sources, including biological sources. In some aspects, the sample is serum, tissue, biopsy tissue, formalin fixed paraffin embedded (FFPE), plasma, saliva, cerebral spinal fluid, tear, urine, synovial fluid, dried blood spot, or any combination thereof.

In some aspects, the analyte is CD73 or a portion thereof. In some aspects, the SIL analyte is V[Ile(¹³C₆, ¹⁵N)]YPAVEGR (SEQ ID NO: 1). In some aspects, the analyte is PD-1 or a portion thereof. In some aspects, the SIL analyte is LAAFPED[Arg(¹³C₆, ¹⁵N₄)] (SEQ ID NO: 2). In some aspects, the analyte is PD-L1 or a portion thereof. In some aspects, the SIL analyte is LQDAG[Val(¹³C₅, ¹⁵N)]YR (SEQ ID NO: 3). In some aspects, the analyte is daclatasvir. In some aspects, the SIL analyte is the SIL analyte is ¹³C₂¹⁵N₄-daclatasvir.

The present methods disclose a composition comprising an In-Sample Calibration Curve (ISCC) wherein ISCC is constructed by multiple isotopologue reaction monitoring (MIRM) of a stable isotopically labeled analyte.

The present methods also disclose a method for quantifying the concentration of at least one analyte in a study sample, the method comprising adding one or more known amount(s) of one or more analyte(s) to a blank matrix sample to construct one or more One-Sample Multipoint External Calibration Curve(s) (OSMECC) by Multiple Isotopologue Reaction Monitoring (MIRM) of each added analyte(s), wherein the MIRM of an analyte refers to multiple reaction monitoring of multiple isotope transitions of the analyte; wherein the OSMECC for each analyte is constructed in the blank matrix sample based on the relationship between the calculated theoretical isotopic abundances (analyte concentration equivalents) in the MIRM transitions and the measured tandem mass spectrometry (MS/MS) peak areas (or peak area ratios if an internal standard is used for the assay) in the corresponding MIRM transitions; wherein the concentration of the at least one analyte in the study sample is quantified using the established OSMECC and the measured peak area (or peak area ratio if an internal standard is used for the assay) for the analyte from a liquid chromatography-tandem mass spectrometry (LC-MS/MS) process; wherein the peak area ratio for the analyte is the peak area of the analyte divided by the peak area of the internal standard, and wherein a tandem mass spectrometer is operated in multiple reaction monitoring mode.

In some aspects, the analyte concentration equivalent for each MIRM transition of the analyte is calculated from a theoretical isotopic abundance of the corresponding MIRM transition of the analyte or the naturally occurring isotopologues of the analyte, wherein the theoretical isotopic abundance is calculated using a methodology published on Analytical Chemistry, 2012, 84(11), 4844-4850, wherein the methodology is calculated based on the isotope distributions of the neutral loss and the daughter ion of the analyte.

In some aspects, for a unit resolution triple quadrupole mass spectrometer, the theoretical isotopic abundance for each of the MIRM transition (m/z) from (p+Zp+α)/Zp to (d+Zd+β)/Zd of the analyte and the naturally occurring isotopologues of the analyte is calculated based on formula (I):

Isotopic abundance in an MIRM transition of (p+Zₚ+α)/Zₚ→(d+Z_{d}+β)/Z_{d}=[relative isotope distribution of the daughter ion at mass of (d+Z_{d}+β)]*[relative isotope distribution of the neutral loss at mass of n+(α−β)] (I)

wherein m/z is the mass to charge ratio,
p is the monoisotopic mass of the parent molecule of the analyte,
Zₚ is the number of charge for the parent ion,
d is the monoisotopic mass of the daughter fragment of the analyte,
Z_{d} is the number of charge for the daughter ion,
n is the monoisotopic mass of the neutral loss of the analyte,
p=d+n,
α and β are integer, α≥0, β≥0 and α≥β, and
Zₚ and Z_{d} are integers

In some aspects, the isotope distribution calculator is at worldwideweb.sisweb.com/mstools/isotope.html (accessed Nov. 10, 2019). In some aspects, the highest analyte concentration equivalent (“Upper Limit of Quantification” or “ULOQ” of the ISCC) is calculated based on formula (II):

(M/V)*ng/mL (IV)

wherein M (ng) is the total amount of the analyte added into the sample;
V is the sample volume (mL) before the analyte is added;

In some aspects, one or more of the other analyte concentration equivalents in the MIRM transitions are calculated based on formula (III):

Iₐ*ULOQ (ng/ml) (III)
Wherein Ia is the calculated theoretical isotopic abundance of a MIRM transition of the analyte or the naturally occurring isotopologues of the analyte.

In some aspects, the analyte is a protein or a peptide. In some aspects, a parent ion of the analyte comprises at least about 3 amino acids, at least about 4 amino acids, at least about 5 amino acids, at least about 6 amino acids, at least about 7 amino acids, at least about 8 amino acids, at least about 9 amino acids, at least about 10 amino acids, at least about 11 amino acids, at least about 12 amino acids, at least about 13 amino acids, at least about 14 amino acids, at least about 15 amino acids, at least about 16 amino acids, at least about 17 amino acids, at least about 18 amino acids, at least about 19 amino acids, or at least about 20 amino acids. In some aspects, a parent ion of the analyte comprises an amino acid sequence between 4 and 20 amino acids, between 4 and 15 amino acids, between 5 and 15 amino acids, between 4 and 14 amino acids, between 5 and 14 amino acids, between 5 and 13 amino acids, between 5 and 12 amino acids, between 6 and 15 amino acids, between 6 and 14 amino acids, between 6 and 13 amino acids, between 6 and 12 amino acids, between 6 and 11 amino acids, between 6 and 10 amino acids, between 6 and 9 amino acids, between 6 and 8 amino acids, between 7 and 15 amino acids, between 7 and 14 amino acids, between 7 and 13 amino acids, between 7 and 12 amino acids, between 7 and 11 amino acids, between 7 and 10 amino acids, or between 7 and 9 amino acids. In some aspects, the analyte is an antibody. In some aspects, the analyte is a fusion protein. In some aspects, the analyte is PD-1, PD-L1, CD73, an anti-PD-1 antibody, an anti-PD-L1 antibody, an anti-CD73 antibody, or any combination thereof. In some aspects, the analyte is a small molecule.

In some aspects, small molecule has a molar mass of at least about 100 g/mol, at least about 200 g/mol, at least about 300 g/mol, at least about 400 g/mol, at least about 500 g/mol, at least about 600 g/mol, at least about 700 g/mol, at least about 800 g/mol, at least about 900 g/mol, at least about 1000 g/mol, at least about 1100 g/mol, at least about 1200 g/mol, at least about 1300 g/mol, at least about 1400 g/mol, at least about 1500 g/mol, at least about 1600 g/mol, at least about 1700 g/mol, at least about 1800 g/mol, at least about 1900 g/mol, or at least about 2000 g/mol. In some aspects, the number of the MIRM transitions is at least two, at least three, at least four, at least five, at least six, at least seven, at least 8, at least 9, at least 10, at least 11, at least 12, at least 13, at least 14, at least 15, at least 16, at least 17, at least 18, at least 19, or at least 20. In some aspects, the number of the MIRM transitions is between 2 and 20. In some aspects, the analyte concentration equivalents of the highest MIRM and the lowest MIRM is at least about 10, at least about 100, at least about 200, at least about 300, at least about 400, at least about 500, at least about 600, at least about 700, at least about 800, at least about 900, at least about 1000, at least about 1100, at least about 1200, at least about 1300, at least about 1400, at least about 1500, at least about 1600, at least about 1700, at least about 1800, at least about 1900, or at least about 2000 fold difference. In some aspects, the calculated theoretical isotopic abundance of two selected MIRM transitions are at least 0.01% apart, at least 0.05% apart, at least 0.1% apart, at least 0.5% apart, at least 1% apart, at least 1.5% apart, at least 2% apart, at least 2.5% apart, at least 3% apart, at least 3.5% apart, at least 4% apart, at least 4.5% apart, at least 5% apart, at least 5.5% apart, at least 6% apart, at least 6.5% apart, at least 7% apart, at least 7.5% apart, at least 8% apart, at least 8.5% apart, at least 9% apart, at least 9.5% apart, at least 10% apart, at least 20% apart, at least 30% apart, at least 40% apart or at least about 50% apart.

### BRIEF DESCRIPTION OF THE DRAWINGS

FIG. 1 shows a scheme diagram for MIRM-ISCC-MS/MS methodology using a surrogate peptide for PD-L1 as an example. The amount of the SIL analyte is added based on the expected concentration of the analyte in order to generate an appropriate calibration curve. Line 1 shows SIL analyte MIRM channel 1 (m/z: 464.2→686.4), isotopic abundance 100%: 100 ng/mL of SIL analyte concentration (99.4 ng/mL of analyte concentration equivalent); Line 2 shows SIL analyte MIRM channel 2 (m/z: 464.7→687.4), isotopic abundance 30.0%: 30.0 ng/mL of SIL analyte concentration (29.8 ng/mL of analyte concentration equivalent); Line 3 shows SIL analyte MIRM channel 3 (m/z: 465.2→688.4), isotopic abundance 6.63%: 6.63 ng/mL of SIL analyte concentration (6.59 ng/mL of analyte concentration equivalent); and Line 4 shows analyte selected reaction monitoring (SRM) channel (m/z: 461.2→680.4), measured concentration: 56.8 ng/mL.

FIGS. 2A and 2B show representative chromatograms for ten MIRM channels of a SIL CD73 peptide, and one selected reaction monitoring (SRM) channel for the CD73 peptide. These ten MIRM channels are used to construct an ISCC for the quantitative analysis of CD73. FIG. 2A is a zoomed out graph; FIG. 2B is a zoomed in graph.

FIG. 3 shows a LC-MS/MS bioanalysis workflow for One-Sample Multipoint External Calibration Curve (OSMECC) and isotope sample dilution.

FIGS. 4A and 4B. FIG. 4A shows a summary of the MRM and MIRM transitions monitored for the multisample external calibration curves, one-sample multipoint external calibration curve (OSMECC), in-sample calibration curve (ISCC) and isotope sample dilution for the measurement of daclatasvir. FIG. 4B shows the calibration curve performances for two multisample external calibration curves and two one-sample multipoint external calibration curves used, as well as two ISCCs.

FIGS. 5A and 5B. FIG. 5A shows the accuracy and precision data for QC samples quantified using multisample external calibration curves, one-sample multipoint external calibration curves, and ISCCs. Both QC samples with concentrations within the calibration curve ranges (1, 3, 40, 500 and 800 ng/mL) and QC samples with concentrations beyond the calibration curve ranges (5000 and 20000 ng/mL) were tested. However, the QC samples at 5000 and 20000 ng/mL were physically diluted (100 and 200-fold respectively) into the calibration curve ranges during the sample preparation. FIG. 5B shows the accuracy and precision data for QC samples quantified using isotope sample dilution. Only QC samples with concentrations beyond the calibration curve ranges (5000, 20000 and 50000 ng/mL) were tested.

### DETAILED DESCRIPTION OF THE DISCLOSURE

The present disclosure provides a highly effective approach to quantify the concentration of an analyte via LC-MS/MS. Specifically, the approach uses an In-Sample Calibration Curve (ISCC) using a stably labeled isotope (SIL) analyte to measure the concentration of an analyte in a sample. A feature of the disclosure is that the ISCC can be used instead of an external calibration curve, thereby reducing the LC-MS/MS total instrument run time. Furthermore, because the ISCC is present inside the sample itself, the present methods eliminate the need of using authentic biological matrix to prepare the external calibration curve and simplifies the quantitative LC-MS/MS bioanalysis process.

As shown in the working examples, the approach is effective at quantifying an analyte concentration via MIRM monitoring of isotope transitions to generate a concentration curve. In certain aspects, the present disclosure provides a method of adding an SIL analyte to a sample containing an analyte wherein the analyte can be quantified via an ISCC generated from “multiple isotopologue reaction monitoring” or “MIRM” of the SIL analyte. In certain aspects, the present disclosure provides a method of analyzing a protein analyte by using a stable isotopically labeled protein or protein fragment. In some aspects, the present disclosure provides a method of quantifying the concentration of an antibody. In other aspects, the present disclosure provides a method of analyzing a small molecule using a stable isotopically labeled variant of the small molecule.

### I. Terms

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety.

The use of the alternative (e.g., “or”) should be understood to mean either one, both, or any combination thereof of the alternatives. As used herein, the indefinite articles “a” or “an” should be understood to refer to “one or more” of any recited or enumerated component.

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within 1 or more than 1 standard deviation per the practice in the art. Alternatively, “about” or “comprising essentially of” can mean a range of up to 10%. Furthermore, particularly with respect to biological systems or processes, the terms can mean up to an order of magnitude or up to 5-fold of a value. When particular values or compositions are provided in the application and claims, unless otherwise stated, the meaning of “about” or “comprising essentially of” should be assumed to be within an acceptable error range for that particular value or composition.

As described herein, any concentration range, percentage range, ratio range or integer range is to be understood to include the value of any integer within the recited range and, when appropriate, fractions thereof (such as one tenth and one hundredth of an integer), unless otherwise indicated.

As used herein, a “biomarker” is virtually any detectable compound, such as a protein, a peptide, a proteoglycan, a glycoprotein, a lipoprotein, a carbohydrate, a lipid, a nucleic acid (e.g., DNA, such as cDNA or amplified DNA, or RNA, such as mRNA), an organic or inorganic chemical, a natural or synthetic polymer, a small molecule (e.g., a metabolite), or a discriminating molecule or discriminating fragment of any of the foregoing, that is present in or derived from a biological sample, or any other characteristic that is objectively measured and evaluated as an indicator of normal biologic processes, pathogenic processes, or pharmacologic responses to a therapeutic intervention, or an indication thereof.

As used herein, “metabolite” refers to any intermediates and products of metabolism. The presence of a drug metabolite is a reliable indicator that a person used the “parent” drug of that metabolite. A metabolite may be a biomarker.

As used herein, “metabolic profile” refers to any defined set of values of quantitative results for metabolites that can be used for comparison to reference values or profiles derived from another sample or a group of samples.

As used herein, the term “isotopologues” refers to a composition that differs from its parent composition in that at least one atom has a different number of neutrons.

As used herein, the term “analyte” is used in its broadest sense to include any molecule or protein (either natural or recombinant), present in a mixture, for which analysis or quantification is desired. Such analytes include, without limitation, small molecules, enzymes, hormones, growth factors, cytokines, peptides, immunoglobulins (e.g., antibodies), and/or any fusion proteins.

As used herein, the terms “isotope” and “isotopologue” are used interchangeably and refer to a molecule with a different isotopic composition as compared to a parent molecule.

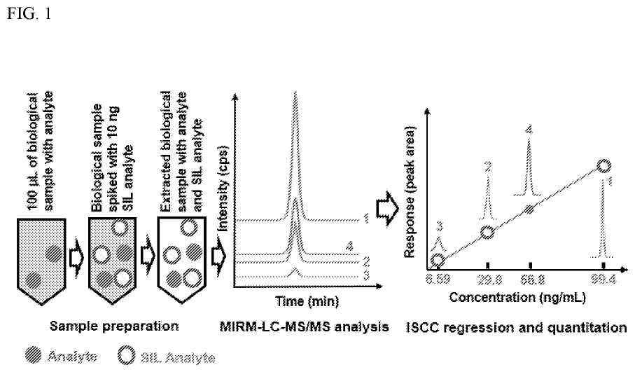

As used herein, the term “analyte equivalent” or “analyte concentration equivalent” refers to the calculated concentrations of the SIL analyte used to construct the In-Sample Calibration Curve (ISCC) after adjusting for the differences in mass between the SIL analyte and the analyte. The ISCC calibration curve is constructed based on measuring MIRMs of the SIL analyte in parallel to determine the peak area of each MIRM. Due to the differences in mass between the SIL analyte and the unlabeled analyte, the calculated concentration curve must be adjusted to account for these minor mass differences, while also adjusting for isotopic abundance. A representative ISCC concentration curve can be seen in FIG. 1, wherein the SIL analyte concentrations are adjusted to account for the differences in mass and isotopic abundance. A sample adjustment to calculate the analyte equivalent for the highest concentration of the concentration curve (Upper limit of quantification “ULOQ”) is represented as point 1 in FIG. 1, and is calculated based on formula (II):

(M/V)*(M_{analyte}/M_{SIL analyte}) ng/mL (II)

Wherein M (ng) is the total amount of the SIL analyte added into the sample;
V is the sample volume (mL) before the SIL analyte is added;
M_{analyte} is the molecular weight of the analyte;
M_{SIL analyte} is the molecular weight of the SIL analyte.

For example, if the SIL analyte is added at a known concentration of 100 ng/mL, and the MIRM has an isotopic abundance of 100%, the analyte concentration equivalent will be 99.4 ng/mL, represented in FIG. 1 as data point 1 and is also the ULOQ. Sample adjustments for the remaining analyte equivalent concentrations of the concentration curve are represented by points 2 and 3 in FIG. 1, and are calculated based on formula (III):

Iₐ*ULOQ (ng/ml) (III)

wherein Iₐ is the calculated theoretical isotopic abundance of a MIRM transition of the SIL analyte or the naturally occurring isotopologues of the SIL analyte.

As used herein, the term “parent ion” or “precursor ion” refers to an electrically charged molecular moiety which may dissociate to form fragments, one or more of which may be electrically charged, and one or more neutral species. A parent ion may be a molecular ion or an electrically charged fragment of a molecular ion.

As used herein, the term “daughter ion” refers to an electrically charged product of reaction of a particular parent (precursor) ion. In general, such ions have a direct relationship with a particular precursor ion and may relate to a unique state of the precursor ion. The reaction need not involve fragmentation, but could, for example involve a change in the number of charges carried. Thus a fragment ion is a daughter ion but not all daughter ions are fragment ions.

As used herein, the term “neutral loss” refers to a mass of neutral charge that is lost during a reaction of a particular parent (precursor) ion during operation of a mass spectrometer.

As used herein, the term “non-peptide molecule” is intended in its broadest sense and can include small molecules and small molecule drugs. A “small molecule” or “small molecule drug” is broadly used herein to refer to an organic, inorganic, or organometallic compound typically having a molecular weight of less than about 1000-2000 g/mol, although this characterization is not intended to be limiting for the purposes of the present invention. “Small Molecule” can also refer to a non-peptidic, non-oligomeric organic compound either synthesized in the laboratory or found in nature. Examples of “small molecules” include, but are not limited to, taxol, clopidogrel, and apixaban. Other examples of small molecules include dapagliflozin, saxagliptin, temsavir, ledipasvir, sofosbuvir, and rosuvastatin.

The term “chromatography” refers to any kind of technique which separates a molecule (e.g., an antibody) from other molecules (e.g., contaminants) present in a mixture. Usually, the molecule is separated from other molecules (e.g., contaminants) as a result of differences in rates at which the individual molecules of the mixture migrate through a stationary medium under the influence of a moving phase, or in bind and elute processes. The term “matrix” or “chromatography matrix” are used interchangeably herein and refer to any kind of sorbent, resin or solid phase which in a separation process separates a molecule from other molecules present in a mixture. Non-limiting examples include particulate, monolithic or fibrous resins as well as membranes that can be put in columns or cartridges. Examples of materials for forming the matrix include polysaccharides (such as agarose and cellulose); and other mechanically stable matrices such as silica (e.g. controlled pore glass), poly(styrenedivinyl)benzene, polyacrylamide, ceramic particles and derivatives of any of the above. Examples for typical matrix types suitable for the method of the present disclosure are cation exchange resins, affinity resins, anion exchange resins or mixed mode resins. A “ligand” is a functional group that is attached to the chromatography matrix and that determines the binding properties of the matrix. Examples of “ligands” include, but are not limited to, ion exchange groups, hydrophobic interaction groups, hydrophilic interaction groups, thiophilic interactions groups, metal affinity groups, affinity groups, bioaffinity groups, and mixed mode groups (combinations of the aforementioned). Some preferred ligands that can be used herein include, but are not limited to, strong cation exchange groups, such as sulphopropyl, sulfonic acid; strong anion exchange groups, such as trimethylammonium chloride; weak cation exchange groups, such as carboxylic acid; weak anion exchange groups, such as N5N diethylamino or DEAE; hydrophobic interaction groups, such as phenyl, butyl, propyl, hexyl; and affinity groups, such as Protein A, Protein G, and Protein L. In order that the present disclosure may be more readily understood, certain terms are first defined. As used in this application, except as otherwise expressly provided herein, each of the following terms shall have the meaning set forth below. Additional definitions are set forth throughout the application.

The term “affinity chromatography” refers to a protein separation technique in which a molecule (e.g., an Fc region containing molecule or antibody) is specifically bound to a ligand which is specific for the molecule. Such a ligand is generally referred to as a biospecific ligand. In some aspects, the biospecific ligand (e.g., Protein A or a functional variant thereof) is covalently attached to a chromatography matrix material and is accessible to the molecule in solution as the solution contacts the chromatography matrix. The molecule generally retains its specific binding affinity for the biospecific ligand during the chromatographic steps, while other solutes and/or proteins in the mixture do not bind appreciably or specifically to the ligand. Binding of the molecule to the immobilized ligand allows contaminating proteins or protein impurities to be passed through the chromatography matrix while the molecule remains specifically bound to the immobilized ligand on the solid phase material. The specifically bound molecule is then removed in active form from the immobilized ligand under suitable conditions (e.g., low pH, high pH, high salt, competing ligand etc.), and passed through the chromatographic column with the elution buffer, free of the contaminating proteins or protein impurities that were earlier allowed to pass through the column. Any component can be used as a ligand for purifying its respective specific binding protein, e.g., antibody. However, in various methods according to the present disclosure, Protein A is used as a ligand for an Fc region containing a target protein. The conditions for elution from the biospecific ligand (e.g., Protein A) of the target protein (e.g., an Fc region containing protein) can be readily determined by one of ordinary skill in the art. In some aspects, Protein G or Protein L or a functional variant thereof may be used as a biospecific ligand. In some aspects, a biospecific ligand such as Protein A is used at a pH range of 5-9 for binding to an Fc region containing protein, washing or re-equilibrating the biospecific ligand/target protein conjugate, followed by elution with a buffer having pH about or below 4 which contains at least one salt.

The terms “purifying,” “separating,” or “isolating,” as used interchangeably herein, refer to increasing the degree of purity of a molecule from a composition or sample comprising the molecule and one or more impurities. Typically, the degree of purity of the molecule is increased by removing (completely or partially) at least one impurity from the composition.

The term “chromatography column” or “column” in connection with chromatography as used herein, refers to a container, frequently in the form of a cylinder or a hollow pillar which is filled with the chromatography matrix or resin. The chromatography matrix or resin is the material which provides the physical and/or chemical properties that are employed for purification.

The terms “ion-exchange” and “ion-exchange chromatography” refer to a chromatographic process in which an ionizable solute of interest (e.g., a molecule in a mixture) interacts with an oppositely charged ligand linked (e.g., by covalent attachment) to a solid phase ion exchange material under appropriate conditions of pH and conductivity, such that the solute of interest interacts non-specifically with the charged compound more or less than the solute impurities or contaminants in the mixture. The contaminating solutes in the mixture can be washed from a column of the ion exchange material or are bound to or excluded from the resin, faster or slower than the solute of interest. “Ion-exchange chromatography” specifically includes cation exchange (CEX), anion exchange (AEX), and mixed mode chromatographies.

A “cation exchange resin” or “cation exchange membrane” refers to a solid phase which is negatively charged, and which has free cations for exchange with cations in an aqueous solution passed over or through the solid phase. Any negatively charged ligand attached to the solid phase suitable to form the cation exchange resin can be used, e.g., a carboxylate, sulfonate and others as described below. Commercially available cation exchange resins include, but are not limited to, for example, those having a sulfonate based group (e.g., MonoS, MiniS, Source 15S and 30S, SP SEPHAROSE® Fast Flow, SP SEPHAROSE® High Performance from GE Healthcare, TOYOPEARL® SP-650S and SP-650M from Tosoh, MACRO-PREP® High S from BioRad, Ceramic HyperD S, TRISACRYL® M and LS SP and Spherodex LS SP from Pall Technologies); a sulfoethyl based group (e.g., FRACTOGEL® SE, from EMD, POROS® S-10 and S-20 from Applied Biosystems); a sulphopropyl based group (e.g., TSK Gel SP 5PW and SP-5PW-HR from Tosoh, POROS® HS-20, HS 50, and POROS® XS from Life Technologies); a sulfoisobutyl based group (e.g., FRACTOGEL® EMD SO₃⁻ from EMD); a sulfoxyethyl based group (e.g., SE52, SE53 and Express-Ion S from Whatman), a carboxymethyl based group (e.g., CM SEPHAROSE® Fast Flow from GE Healthcare, Hydrocell CM from Biochrom Labs Inc., MACRO-PREP® CM from BioRad, Ceramic HyperD CM, TRISACRYL® M CM, TRISACRYL® LS CM, from Pall Technologies, Matrx CELLUFINE® C500 and C200 from Millipore, CM52, CM32, CM23 and Express-Ion C from Whatman, TOYOPEARL® CM-650S, CM-650M and CM-650C from Tosoh); sulfonic and carboxylic acid based groups (e.g., BAKERBOND® Carboxy-Sulfon from J. T. Baker); a carboxylic acid based group (e.g., WP CBX from J. T Baker, DOWEX®. MAC-3 from Dow Liquid Separations, AMBERLITE® Weak Cation Exchangers, DOWEX® Weak Cation Exchanger, and DIAION® Weak Cation Exchangers from Sigma-Aldrich and FRACTOGEL® EMD COO—from EMD); a sulfonic acid based group (e.g., Hydrocell SP from Biochrom Labs Inc., DOWEX® Fine Mesh Strong Acid Cation Resin from Dow Liquid Separations, UNOsphere S, WP Sulfonic from J. T. Baker, SARTOBIND® S membrane from Sartorius, AMBERLITE® Strong Cation Exchangers, DOWEX® Strong Cation and DIAION® Strong Cation Exchanger from Sigma-Aldrich); or a orthophosphate based group (e.g., P11 from Whatman).

Other cation exchange resins include Poros HS, Poros XS, carboxy-methyl-cellulose, BAKERBOND ABX™, sulphopropyl immobilized on agarose and sulphonyl immobilized on agarose, MonoS, MiniS, Source 15S, 30S, SP SEPHAROSE™, CM SEPHAROSE™, BAKERBOND Carboxy-Sulfon, WP CBX, WP Sulfonic, Hydrocell CM, Hydrocel SP, UNOsphere S, Macro-Prep High S, Macro-Prep CM, Ceramic HyperD S, Ceramic HyperD CM, Ceramic HyperD Z, Trisacryl M CM, Trisacryl LS CM, Trisacryl M SP, Trisacryl LS SP, Spherodex LS SP, DOWEX Fine Mesh Strong Acid Cation Resin, DOWEX MAC-3, Matrex Cellufine C500, Matrex Cellufine C200, Fractogel EMD S03-, Fractogel EMD SE, Fractogel EMD COO—, Amberlite Weak and Strong Cation Exchangers, Diaion Weak and Strong Cation Exchangers, TSK Gel SP-5PW-HR, TSK Gel SP-5PW, Toyopearl CM (650S, 650M, 650C), Toyopearl SP (650S, 650M, 650C), CM (23, 32, 52), SE(52, 53), P11, Express-Ion C or Express-Ion S.

An “anion exchange resin” or “anion exchange membrane” refers to a solid phase which is positively charged, thus having one or more positively charged ligands attached thereto. Any positively charged ligand attached to the solid phase suitable to form the anionic exchange resin can be used, such as quaternary amino groups. Commercially available anion exchange resins include DEAE cellulose, POROS® PI 20, PI 50, HQ 10, HQ 20, HQ 50, D 50 from Applied Biosystems, SARTOBIND® Q from Sartorius, MonoQ, MiniQ, Source 15Q and 30Q, Q, DEAE and ANX SEPHAROSE® Fast Flow, Q SEPHAROSE® High Performance, QAE SEPHADEX® and FAST Q SEPHAROSE® (GE Healthcare), WP PEI, WP DEAM, WP QUAT from J. T. Baker, Hydrocell DEAE and Hydrocell QA from Biochrom Labs Inc., UNOsphere Q, MACRO-PREP®. DEAE and MACRO-PREP® High Q from Biorad, Ceramic HyperD Q, ceramic HyperD DEAE, TRISACRYL® M and LS DEAE, Spherodex LS DEAE, QMA SPHEROSIL® LS, QMA SPHEROSIL®. M and MUSTANG® Q from Pall Technologies, DOWEX® Fine Mesh Strong Base Type I and Type II Anion Resins and DOWEX® MONOSPHERE 77, weak base anion from Dow Liquid Separations, INTERCEPT® Q membrane, Matrex CELLUFINE® A200, A500, Q500, and Q800, from Millipore, FRACTOGEL® EMD TMAE, FRACTOGEL® EMD DEAE and FRACTOGEL® EMD DMAE from EMD, AMBERLITE® weak strong anion exchangers type I and II, DOWEX® weak and strong anion exchangers type I and II, DIAION® weak and strong anion exchangers type I and II, DUOLITE® from Sigma-Aldrich, TSK gel Q and DEAE 5PW and 5PW-HR, TOYOPEARL® SuperQ-650S, 650M and 650C, QAE-550C and 650S, DEAE-650M and 650C from Tosoh, QA52, DE23, DE32, DE51, DE52, DE53, Express-Ion D or Express-Ion Q from Whatman, and SARTOBIND® Q (Sartorius Corporation, New York, USA).

Other anion exchange resins include POROS HQ, Q SEPHAROSE™ Fast Flow, DEAE SEPHAROSE™ Fast Flow, SARTOBIND® Q, ANX SEPHAROSE™ 4 Fast Flow (high sub), Q SEPHAROSE™ XL, Q SEPHAROSE™ big beads, DEAE Sephadex A-25, DEAE Sephadex A-50, QAE Sephadex A-25, QAE Sephadex A-50, Q SEPHAROSE™ high performance, Q SEPHAROSE™ XL, Sourse 15Q, Sourse 30Q, Resourse Q, Capto Q, Capto DEAE, Mono Q, Toyopearl Super Q, Toyopearl DEAE, Toyopearl QAE, Toyopearl Q, Toyopearl GigaCap Q, TS gel SuperQ, TS gel DEAE, Fractogel EMD TMAE, Fractogel EMD TMAE HiCap, Fractogel EMD DEAE, Fractogel EMD DMAE, Macroprep High Q, Macro-prep-DEAE, Unosphere Q, Nuvia Q, PORGS PI, DEAE Ceramic HyperD, or Q Ceramic HyperD.

“Mass spectrometry” (“MS” or “mass-spec”) is an analytical technique used to measure the mass-to-charge ratio ions. This is achieved by ionizing the sample and separating ions of differing masses and recording their relative abundance by measuring intensities of ion flux. A typical mass spectrometer comprises three parts: an ion source, a mass analyzer, and a detector system. The ion source is the part of the mass spectrometer that ionizes the substance under analysis (the analyte). The ions are then transported by magnetic or electric fields to the mass analyzer that separates the ions according to their mass-to-charge ratio (m/z). Many mass spectrometers use two or more mass analyzers for tandem mass spectrometry (MS/MS). The detector records the charge induced or current produced when an ion passes by or hits a surface. A mass spectrum is the result of measuring the signal produced in the detector when scanning m/z ions with a mass analyzer.

The term “In-Sample Calibration Curve (ISCC)” as used herein refers to a calibration curve that is present in the sample matrix itself rather than as, for example, an external calibration curve traditionally used to perform LC-MS/MS analysis.

The term “SIL analyte” or “stable isotopically labeled analyte” as used herein refers to a compound that is an analyte or a fragment thereof that has been modified to contain an isotopic element at one or more positions. The most common labeling technique uses ¹³C or ⁵N as the stable isotope, and the compound can be labeled at one or more positions. Other suitable stable isotopes include ²H, ³³S, ³⁴S, ³⁶S, ¹⁷O, or ¹⁸O.

The term “multiple isotopologue reaction monitoring” or MIRM refers to the process by which the mass spectrometer is tuned to monitor particular multiple reaction monitoring (MRM) transitions from a parent ion and its isotopologue ions (the parent ion with different neutrons) to their product ions with the same cleavage site, also referred to as “MIRM channels” or “MIRM transitions”. More generally, when using a mass spectrometer to measure a single ion alone, the process is also called selected reaction monitoring (SRM). When multiple reactions are measured via SRM wherein multiple product ions are produced from one or more precursor ions, this process is known in the art as multiple reaction monitoring (MRM). In a SRM experiment on a triple-quadrupole mass spectrometer, the first quadrupole (Q1) is set to pass ions only of a specified m/z (precursor ions) of an expected chemical species in the sample. The second quadrupole (i.e. Q2 or the collision cell) is used to fragment the ions passing through Q1. The third quadrupole (Q3) is set to pass to the detector only ions of a specified m/z (fragment ions) corresponding to an expected fragmentation product of the expected chemical species. When numerous SRM experiments are run, the process is called Multiple Reaction Monitoring (“MRM”).

As used herein, the term “MIRM transition” or alternately, the parent-daughter ion transition pair “PDITP” refers to the pair of m/z values being monitored. Briefly, for a parent ion P (monoisotopic mass of p+1.00783*Zₚ, Zₚ is the number of charge for the parent ion and hydrogen monoisotopic mass is 1.00783 Da) with a daughter ion D (monoisotopic mass of d+1.00783*Z_{d}, Z_{d} is the number of charge for the daughter ion) and neutral loss N (monoisotopic mass of n), the most abundant (100%) MIRM channel (m/z) is shown below:

(p+1.00783*Zₚ)/Zₚ→(d+1.00783*Z_{d})/Z_{d}

For a unit resolution triple quadrupole mass spectrometer using most commonly used charge states (singly-, doubly- and triply-charged ions), this MIRM channel (m/z) can be simplified as:

(p+Zₚ)/Zₚ→(d+Z_{d})/Z_{d}

The isotopic abundance in an adjacent MIRM channel (m/z) of

(p+Zₚ+α)/Zₚ→(d+Z_{d}+β)/Z_{d}

can be calculated as:

Isotopic abundance in an MIRM transition of (p+Zₚ+α)/Zₚ→(d+Z_{d}+β)/Z_{d}=[relative isotope distribution of the daughter ion at mass of (d+Z_{d}+β)]*[relative isotope distribution of the neutral loss at mass of n+(α−β)]

where: (1) p=d+n
(2) α and β are integers, they are the number of additional neutrons on the parent ion and daughter ion, respectively, α≥0, β≥0 and α≥≥
(3) Zₚ and Z_{d} are integers
(4) Isotopic distribution of a molecule can be found using an online calculator (worldwideweb.sisweb.com/mstools/isotope.html, accessed Nov. 10, 2019)
(5) Relative isotopic distributions of the daughter ion and neutral loss at mass of d (α=0) and n (α−β=0), respectively, are 100%.

By using different combinations of α and β, the isotopic abundances in different adjacent MIRM channels (m/z) of (p+Zₚ+α)/Zₚ→(d+Z_{d}+β)/Z_{d} can be calculated and measured accurately.

Various aspects of the disclosure are described in further detail in the following subsections.

### II. Methods of Quantifying

The present disclosure is directed to a Multiple Isotopologue Reaction Monitoring-In Sample Curve Calibration-LC-MS/MS (MIRM-ISCC-LC-MS/MS) methodology, which allows the instant and accurate measurement of each individual sample without using external calibration curves, thus eliminating the need of using authentic biological matrix, simplifying the quantitative LC-MS/MS bioanalysis process, and greatly reducing instrument time. While MIRM-ISCC-LC-MS/MS methodology can be applied in regular pharmacokinetic (PK) sample analysis in drug discovery and development, this methodology is particularly useful for cases where authentic matrix is hardly available, such as biomarker measurement and quantitative proteomics, where the low throughput and long turnaround time are the main issues preventing the use of LC-MS/MS technique, such as the clinical diagnosis in clinical diagnostic laboratories, and where calibration curve preparation is cumbersome, such as the fresh frozen and FFPE tissue analysis. Additionally, an ISCC can also be used as an external calibration curve by spiking a known amount of non-labeled analyte in blank matrix, and therefore, an external calibration curve can be constructed in just one sample, eliminating the need for preparation of multiple samples for an external calibration curve.

Normally, the most abundant MIRM channel of an analyte is monitored in a LC-MS/MS assay for quantitative analysis. Due to an elements' naturally occurring isotopes, in addition to the MS/MS response observed in its most abundant MIRM channel, isotopic abundances in isotope MIRM channels adjacent to the most abundant MIRM channel can be accurately calculated and measured by LC-MS/MS. Briefly, for a parent ion P (monoisotopic mass of p+0.00783*Zₚ, Zₚ is the number of charge for the parent ion and hydrogen monoisotopic mass is 1.00783 Da) with a daughter ion D (monoisotopic mass of d+1.00783*Z_{d}, Z_{d} is the number of charge for the daughter ion) and neutral loss N (monoisotopic mass of n), the most abundant (100%) MIRM channel (m/z) is shown below:

(p+1.00783*Zₚ)/Zₚ→(d+1.00783*Z_{d})/Z_{d}

For a unit resolution triple quadrupole mass spectrometer using most commonly used charge states (singly-, doubly- and triply-charged ions), this MIRM channel (m/z) can be simplified as:

(p+Zₚ)/Zₚ→(d+Z_{d})/Z_{d}

The isotopic abundance in an adjacent MIRM channel (m/z) of

(p+Zₚ+α)/Zₚ→(d+Z_{d}+β)/Z_{d}

can be calculated as:

Isotopic abundance in an MIRM transition of (p+Zₚ+α)/Zₚ→(d+Z_{d}+β)/Z_{d}=[relative isotope distribution of the daughter ion at mass of (d+Z_{d}+β)]*[relative isotope distribution of the neutral loss at mass of n+(α−β)]

where: (1) p=d+n
(2) α and β are integer, they are the number of additional neutrons on the parent ion and daughter ion, respectively, α≥0, β≥0 and α≥β
(3) Zₚ and Z_{d} are integers
(4) Isotopic distribution of a molecule can be found using an online calculator (worldwideweb.sisweb.com/mstools/isotope.html (accessed Nov. 10, 2019)
(5) Relative isotopic distributions of the daughter ion and neutral loss at mass of d (α=0) and n (α−β=0), respectively, are 100%

By using different combinations of α and β, the isotopic abundances in different adjacent MIRM channels (m/z) of (p+Zₚ+α)/Zₚ→(d+Z_{d}+β)/Z_{d} can be calculated and measured accurately. The isotopic abundances from the most abundant MIRM channel (α=0 and β=0) to the lowest abundant MIRM channel could reach as high as 5 to 6 orders of magnitude. In theory, as these isotopic abundances are coming from the combinations of different isotopes of the daughter ion and neutral loss, and they have “identical” physicochemical properties, a linear relationship should exist between the MS/MS responses (peak areas) and the calculated theoretical isotopic abundances in all of the adjacent MIRM channels over the entire range. However, this linear relationship is limited by the mass spectrometer's detection limit as well as the linear range of the most mass spectrometers. To maintain this linear relationship, it is necessary to set the same mass spectrometer parameters, such as dwell time, collision energy (CE) and declustering potential (DP) etc., for all MIRM channels used for the SIL analyte and the SRM channel used for the analyte.

The present methods demonstrate a completely new methodology that uses isotopic abundances in Multiple Isotopologue Reaction Monitoring (MIRM) channels to construct an In-Sample Calibration Curve (ISCC) in every study sample for quantitative LC-MS/MS bioanalysis. There are numerous potential applications of MIRM-ISCC-LC-MS/MS methodology such as use in quantitative analysis of small molecules, peptides, proteins, biomarkers, quantitative proteomics, clinical diagnostic laboratories and other areas.

Absolute quantitation in LC-MS proteomics with isotope dilution principle was achieved by spiking a known amount of a SIL peptide (AQUA approach) or protein (PSAQ approach) into each study sample. The peptide (or protein) concentration of a study sample could be calculated using the ratio between the peak area of the non-labeled peptide (or protein) and the peak area of the labeled peptide (or protein). However, this quantitation approach is based on only one calibration point with the assumption that a linear relationship passing through the point of origin exists between the MS responses (or response ratios) and the corresponding concentrations.

Recently, to address this issue, Chiva et al. (66th ASMS Conference on Mass Spectrometry and Allied Topics, Jun. 3-7, 2018, San Diego, Calif., p ThP 481) reported that an accurate and robust assay was developed by spiking a calibration curve into each study sample for the absolute quantitation of a targeted peptide in Formalin Fixed Paraffin Embedded (FFPE) samples. This calibration curve was pre-prepared using five different SIL peptide analytes (with total labeling positions of 10, 16, 23, 33 and 39 for each SIL peptide analyte, respectively) at different concentration levels from 2 to 200 fmol. However, as multiple differently labeled peptide analytes with as many as 30 to 40 labeling positions are needed for the analysis of one targeted peptide, this approach is very costly and time consuming especially for quantitative proteomics of multiple targeted peptides.

The methods useful in the present disclosure involve detecting the presence of and/or quantifying the concentration of at least one analyte in a sample, the method comprising adding one or more known amount(s) stable isotopically labeled (SIL) analyte(s) to a sample containing at least one analyte to construct one or more In-Sample Calibration Curve(s) (ISCC) by Multiple Isotopologue Reaction Monitoring (MIRM) of each added SIL analyte(s), wherein the MIRM of an SIL analyte refers to multiple reaction monitoring of multiple isotope transitions of the SIL analyte; wherein the ISCC for each analyte is constructed in the sample based on the relationship between the calculated theoretical isotopic abundances (analyte concentration equivalents) in the MIRM transitions and the measured tandem mass spectrometry (MS/MS) peak areas in the corresponding MIRM transitions; wherein the concentration of the at least one analyte in the sample is quantified using the established ICSS and the measured peak area for the analyte from a liquid chromatography-tandem mass spectrometry (LC-MS/MS) process, and wherein a tandem mass spectrometer is operated in multiple reaction monitoring mode.

In some aspects, the analyte, the SIL analyte, and the naturally occurring isotopologues of the SIL analyte are ionized in the mass spectrometer to produce protonated (or deprotonated) parent ions of the analyte, the SIL analyte and the naturally occurring isotopologues of the SIL analyte. In some aspects, the parent ions of the analyte, the parent ions of the SIL analyte, and the parent ions of the naturally occurring isotopologues of the SIL analyte in the mass spectrometer are fragmented at the same cleavage site to produce neutral losses and daughter ions.

In some aspects, the transition from the parent ion to the daughter ion for the analyte is monitored in the mass spectrometer and a peak area for the transition from the parent ion to the daughter ion for the analyte is measured. In some aspects, the selected multiple transitions from the parent ions of the SIL analyte and the parent ions of the naturally occurring isotopologues of the SIL analyte to the daughter ions of the SIL analyte and the daughter ions of the naturally occurring isotopologues of the SIL analyte are monitored in the mass spectrometer (“multiple isotopologue reaction monitoring” or “MIRM”);

In some aspects, a peak area of each of the MIRM transitions is measured, wherein the MIRM transitions comprise the selected transitions from parent ions of the SIL analyte and the parent ions of the naturally occurring isotopologues of the SIL analyte to the daughter ions of the SIL analyte and the daughter ions of the naturally occurring isotopologues of the SIL analyte.

In some aspects, an In-Sample Calibration Curve is generated based on the relationship between the measured peak areas in the MIRM transitions of the SIL analyte and the naturally occurring isotopologues of the SIL analyte, and the analyte concentration equivalents for each of the MIRM transitions.

In some aspects, the analyte concentration equivalent for each MIRM transition is calculated from a theoretical isotopic abundance of the corresponding MIRM transition of the SIL analyte or the naturally occurring isotopologues of the SIL analyte, wherein the theoretical isotopic abundance is calculated using a methodology published in Analytical Chemistry, 2012, 84(11), 4844-4850, wherein the methodology is calculated based on the isotope distributions of the neutral loss and the daughter ion of the SIL analyte. In other aspects, the theoretical isotopic abundance for each of the MIRM transition (m/z) from (p+Zₚ+α)/Zₚ to (d+Z_{d}+β)/Z_{d} of the SIL analyte and the naturally occurring isotopologues of the SIL analyte is calculated based on formula (I):

Isotopic abundance in an MIRM transition of (p+Zₚ+α)/Zₚ→(d+Z_{d}+β)/Z_{d}=[relative isotope distribution of the daughter ion at mass of (d+Z_{d}+β)]*[relative isotope distribution of the neutral loss at mass of n+(α−β)] (I)

Wherein m/z is the mass to charge ratio
p is the monoisotopic mass of the parent molecule of the SIL analyte
Zₚ is the number of charge for the parent ion
d is the monoisotopic mass of the daughter fragment of the SIL analyte
Z_{d} is the number of charge for the daughter ion
n is the monoisotopic mass of the neutral loss of the SIL analyte
β=d+n
α and β are integer, they are the number of additional neutrons on the parent ion and daughter ion, respectively, α≥0, β≥0 and α≥β
Zₚ and Z_{d} are integers

In some aspects, the isotopic abundance calculator can be found at worldwideweb.sisweb.com/mstools/isotope.html (accessed Nov. 10, 2019). In some aspects, wherein the highest analyte concentration equivalent (“Upper Limit of Quantification” or “ULOQ” of the ISCC) is calculated based on formula (II):

(M/V)*(M_{analyte}/M_{SIL analyte}) ng/mL (II)

Wherein M (ng) is the total amount of the SIL analyte added into the sample;
V is the sample volume (mL) before the SIL analyte is added;
M_{analyte} is the molecular weight of the analyte;
M_{SIL analyte} is the molecular weight of the SIL analyte.

In some aspects, one or more of the other analyte concentration equivalents in the MIRM transitions are calculated based on formula (III):

Iₐ*ULOQ (ng/ml) (III)
wherein Iₐ is the calculated theoretical isotopic abundance of a MIRM transition of the SIL analyte or the naturally occurring isotopologues of the SIL analyte.

The method useful in the present disclosure involves the use of a stable isotopically labeled (SIL) analyte that is added to a sample containing an analyte. This SIL analyte can then be monitored via MIRM technique to construct a concentration calibration curve to quantify the concentration of an analyte present in the sample. In some aspects, the analyte is a protein or a peptide and the SIL analyte is a stable isotopically labeled protein or peptide. In some aspects, a parent ion of the SIL analyte comprises at least about 3 amino acids, at least about 4 amino acids, at least about 5 amino acids, at least about 6 amino acids, at least about 7 amino acids, at least about 8 amino acids, at least about 9 amino acids, at least about 10 amino acids, at least about 11 amino acids, at least about 12 amino acids, at least about 13 amino acids, at least about 14 amino acids, at least about 15 amino acids, at least about 16 amino acids, at least about 17 amino acids, at least about 18 amino acids, at least about 19 amino acids, or at least about 20 amino acids, at least about 21 amino acids, at least about 22 amino acids, at least about 23 amino acids, at least about 24 amino acids, at least about 25 amino acids, at least about 26 amino acids, at least about 27 amino acids, at least about 28 amino acids, at least about 29 amino acids, at least about 30 amino acids, at least about 31 amino acids, at least about 32 amino acids, at least about 33 amino acids, at least about 34 amino acids, at least about 35 amino acids, at least about 36 amino acids, at least about 37 amino acids, at least about 38 amino acids, at least about 39 amino acids, or at least about 40 amino acids.

In some aspects, a parent ion of the SIL analyte comprises an amino acid sequence between 4 and 40 amino acids, between 4 and 35 amino acids, between 5 and 35 amino acids, between 4 and 34 amino acids, between 5 and 34 amino acids, between 5 and 33 amino acids, between 5 and 32 amino acids, between 6 and 35 amino acids, between 6 and 34 amino acids, between 6 and 33 amino acids, between 6 and 32 amino acids, between 6 and 31 amino acids, between 6 and 30 amino acids, between 6 and 29 amino acids, between 6 and 28 amino acids, between 7 and 35 amino acids, between 7 and 34 amino acids, between 7 and 33 amino acids, between 7 and 32 amino acids, between 7 and 31 amino acids, between 7 and 30 amino acids, or between 7 and 29 amino acids. In some aspects, a parent ion of the analyte or the SIL analyte comprises an amino acid sequence between 7 and 11 amino acids. In some aspects, a parent ion of the SIL analyte comprises an amino acid sequence between 8 and 11 amino acids. In some aspects, a parent ion of the SIL analyte comprises an amino acid sequence between 8 and 10 amino acids. In some aspects, a parent ion of the SIL analyte comprises an amino acid sequence between 8 and 9 amino acids. In some aspects, a parent ion of the SIL analyte comprises an amino acid sequence between 6 and 9 amino acids. In some aspects, a parent ion of the SIL analyte comprises an amino acid sequence between 6 and 10 amino acids.

In some aspects, a parent ion of the SIL analyte comprises an amino acid sequence between 4 and 30 amino acids, between 4 and 25 amino acids, between 5 and 25 amino acids, between 4 and 24 amino acids, between 5 and 24 amino acids, between 5 and 23 amino acids, between 5 and 22 amino acids, between 6 and 25 amino acids, between 6 and 24 amino acids, between 6 and 23 amino acids, between 6 and 22 amino acids, between 6 and 21 amino acids, between 6 and 20 amino acids, between 7 and 25 amino acids, between 7 and 24 amino acids, between 7 and 23 amino acids, between 7 and 22 amino acids, between 7 and 21 amino acids, or between 7 and 20 amino acids.

In some aspects, a parent ion of the SIL analyte comprises an amino acid sequence between 4 and 20 amino acids, between 4 and 15 amino acids, between 5 and 15 amino acids, between 4 and 14 amino acids, between 5 and 14 amino acids, between 5 and 13 amino acids, between 5 and 12 amino acids, between 6 and 15 amino acids, between 6 and 14 amino acids, between 6 and 13 amino acids, between 6 and 12 amino acids, between 6 and 11 amino acids, between 6 and 10 amino acids, between 6 and 9 amino acids, between 6 and 8 amino acids, between 7 and 15 amino acids, between 7 and 14 amino acids, between 7 and 13 amino acids, between 7 and 12 amino acids, between 7 and 11 amino acids, between 7 and 10 amino acids, or between 7 and 9 amino acids.

In some aspects, the SIL analyte is a stable isotopically labeled protein or peptide. In some aspects, a parent ion of the SIL analyte comprises at least about 3 amino acids, at least about 4 amino acids, at least about 5 amino acids, at least about 6 amino acids, at least about 7 amino acids, at least about 8 amino acids, at least about 9 amino acids, at least about 10 amino acids, at least about 11 amino acids, at least about 12 amino acids, at least about 13 amino acids, at least about 14 amino acids, at least about 15 amino acids, at least about 16 amino acids, at least about 17 amino acids, at least about 18 amino acids, at least about 19 amino acids, or at least about 20 amino acids.

In some aspects, the analyte is an antibody. In other aspects, the analyte is a fusion protein. In some aspects, the analyte is a fusion protein comprising a protein and a heterologous moiety. In other aspects, the analyte is an Fc fusion protein. In some aspects, the analyte is PD-1, PD-L1, CD73, an anti-PD-1 antibody, an anti-PD-L1 antibody, an anti-CD73 antibody, or any combination thereof. In some aspects, the analyte is an anti-GITR antibody, an anti-CXCR4 antibody, an anti-TIGIT antibody, an anti-OX40 antibody, an anti-LAG3 antibody, an anti-TIM3 antibody, an anti-IL8 antibody, or any combination thereof.

The present methods are effective to detect or quantify an analyte that is a protein using a corresponding SIL analyte. In some aspects, the analyte is an antibody. In some aspects, the analyte is CD73 or an anti-CD73 antibody or fragment thereof. In some aspects, the analyte is PD-1 or an anti-PD-1 antibody such as nivolumab, or a fragment thereof. In some aspects, the analyte is PD-L1 or an anti-PD-L1 antibody such as ipilimumab, or a fragment thereof. In some aspects, the analyte is an anti-OX40 (also known as CD134, TNFRSF4, ACT35 and/or TXGP1L) antibody (e.g., BMS986178, or MDX-1803), or a fragment thereof. In some aspects, the analyte is ulocuplumab, or a fragment thereof. In some aspects, the analyte is BMS-986156, or a fragment thereof. In some aspects, the analyte is BMS-986016, or a fragment thereof. In some aspects, the analyte is BMS-986207, or a fragment thereof. In some aspects, the analyte BMS-986253, or a fragment thereof. In some aspects, the analyte BMS-986258, or a fragment thereof.

In some aspects, the analyte is an anti-PD-1 antibody. In some aspects, the anti-PD-1 antibody is selected from the group consisting of nivolumab (also known as OPDIVO®, 5C4, BMS-936558, MDX-1106, and ONO-4538), pembrolizumab (Merck; also known as KEYTRUDA®, lambrolizumab, and MK-3475; see WO2008/156712), PDR001 (Novartis; see WO 2015/112900), MEDI-0680 (AstraZeneca; also known as AMP-514; see WO 2012/145493), cemiplimab (Regeneron; also known as REGN-2810; see WO 2015/112800), JS001 (TAIZHOU JUNSHI PHARMA; see Si-Yang Liu et al., J. Hematol. Oncol. 10:136 (2017)), BGB-A317 (Beigene; see WO 2015/35606 and US 2015/0079109), INCSHR1210 (Jiangsu Hengrui Medicine; also known as SHR-1210; see WO 2015/085847; Si-Yang Liu et al., J. Hematol. Oncol. 10:136 (2017)), TSR-042 (Tesaro Biopharmaceutical; also known as ANBO11; see WO2014/179664), GLS-010 (Wuxi/Harbin Gloria Pharmaceuticals; also known as WBP3055; see Si-Yang Liu et al., J. Hematol. Oncol. 10:136 (2017)), AM-0001 (Armo), STI-1110 (Sorrento Therapeutics; see WO 2014/194302), AGEN2034 (Agenus; see WO 2017/040790), MGA012 (Macrogenics, see WO 2017/19846), and IBI308 (Innovent; see WO 2017/024465, WO 2017/025016, WO 2017/132825, and WO 2017/133540).

In certain aspects, the analyte is an anti-PD-L1 antibody. In some aspects, the anti-PD-L1 antibody is selected from the group consisting of BMS-936559 (also known as 12A4, MDX-1105; see, e.g., U.S. Pat. No. 7,943,743 and WO 2013/173223), atezolizumab (Roche; also known as TECENTRIQ®; MPDL3280A, RG7446; see U.S. Pat. No. 8,217,149; see, also, Herbst et al. (2013) J Clin Oncol 31(suppl):3000), durvalumab (AstraZeneca; also known as IMFINZI™, MEDI-4736; see WO 2011/066389), avelumab (Pfizer; also known as BAVENCIO®, MSB-0010718C; see WO 2013/079174), STI-1014 (Sorrento; see WO2013/181634), CX-072 (Cytomx; see WO2016/149201), KNO35 (3D Med/Alphamab; see Zhang et al., CellDiscov. 7:3 (March 2017), LY3300054 (Eli Lilly Co.; see, e.g., WO 2017/034916), and CK-301 (Checkpoint Therapeutics; see Gorelik et al., AACR:Abstract 4606 (April 2016)).

In some aspects, the analyte is CD73 or a portion thereof. In some aspects, the SIL analyte is V[Ile(¹³C₆, ¹⁵N)]YPAVEGR (SEQ ID NO: 1). In some aspects, the analyte is PD-1 or a portion thereof. In some aspects, the SIL analyte is LAAFPED[Arg(¹³C₆, ¹⁵N₄)](SEQ ID NO: 2). In some aspects, the analyte is PD-L1 or a portion thereof. In some aspects, the SIL analyte is LQDAG[Val(¹³C₅, ¹⁵N)]YR (SEQ ID NO: 3). In some aspects, the analyte is daclatasvir. In some aspects, the SIL analyte is the SIL analyte is ¹³C₂¹⁵N₄-daclatasvir.

In some aspects, the analyte is a non-peptide molecule. In some aspects, the analyte has a molecular weight of at least 100 g/mol, of at least 200 g/mol, of at least 300 g/mol, of at least 400 g/mol, of at least 500 g/mol, of at least 600 g/mol, of at least 700 g/mol, of at least 800 g/mol, of at least 900 g/mol, of at least 1000 g/mol, of at least 1100 g/mol, of at least 1200 g/mol, of at least 1300 g/mol, of at least 1400 g/mol, of at least 1500 g/mol, of at least 1600 g/mol, of at least 1700 g/mol, of at least 1800 g/mol, of at least 1900 g/mol, or of at least 2000 g/mol.

In some aspects, the analyte is an anti-bacterial agent or an anti-viral agent. In some aspects, the analyte is an agent against hepatitis B, hepatitis C, HIV, syphilis, or any combination thereof. In other aspects, the analytes having anti-HCV activity are those that are effective to inhibit the function of a target selected from HCV metalloprotease, HCV serine protease, HCV polymerase, HCV helicase, HCV NS4B protein, HCV entry, HCV assembly, HCV egress, HCV NS5A protein and IMPDH, and/or cyclosporine analogs and/or a nucleoside analog for the treatment of an HCV or flaviviridae infection.

Among the analytes that can be used in the present disclosure, as selective HCV serine protease inhibitors, are the peptide compounds disclosed in Patent No. WO/1999/007733, WO/2005/007681, WO/2005/028502, WO/2005/035525, WO/2005/037860, WO/2005/077969, WO/2006/039488, WO/2007/022459, WO/2008/106058, WO 2008/106139, WO/2000/009558, WO/2000/009543, WO/1999/064442, WO/1999/007733, WO/1999/07734, WO/1999/050230 and WO/1998/017679. NS5B polymerase inhibitors have also demonstrated activity. These agents include but are not limited to other inhibitors of HCV RNA dependent RNA polymerase such as, for example, nucleoside type polymerase inhibitors described in WO01/90121(A2), or U.S. Pat. No. 6,348,587B1 or WO01/60315 or WO01/32153 or non-nucleoside inhibitors such as, benzimidazole polymerase inhibitors described in EP 162196A1 or WO02/04425.

In addition to the combinations of pegylated alpha-interferon and ribavirin, other combinations of compounds useful for treating HCV-infected patients are desired which selectively inhibit HCV viral replication. In particular, pharmaceutical agents which are effective to inhibit the function of the NS5A protein in combination with those effective to inhibit other viral targets are desired. The HCV NS5A protein is described, for example, in Tan, S.-L.; Katzel, M. G. Virology (2001) 284, 1-12, and in Park, K.-J.; Choi, S.-H, J. Biological Chemistry (2003). The relevant patent disclosures describing the synthesis of HCV NS5A inhibitors are: US 2009/0202478; US 2009/0202483; WO 2009/020828; WO 2009/020825; WO 2009/102318; WO 2009/102325; WO 2009/102694; WO 2008/144380; WO 2008/021927; WO 2008/021928; WO 2008/021936; WO 2006/133326; WO 2004/014852; WO 2008/070447; WO 2009/034390; WO 2006/079833; WO 2007/031791; WO 2007/070556; WO 2007/070600; WO 2008/064218; WO 2008/154601; WO 2007/082554; WO 2008/048589; WO 2010/017401; WO 2010/065668; WO 2010/065674; WO 2010/065681, the contents of each of which are expressly incorporated by reference herein.

In some aspects, the analyte is a small molecule. In some aspects, the analyte is taxol, clopidogrel, apixaban, dapagliflozin, saxagliptin, temsavir, ledipasvir, sofosbuvir, or rosuvastatin. In some aspects, the analyte is taxol. In some aspects, the analyte is clopidogrel. In some aspects, the analyte is apixaban. In some aspects, the analyte is dapagliflozin. In some aspects, the analyte is saxagliptin. In some aspects, the analyte is temsavir. In some aspects, the analyte is ledipasvir. In some aspects, the analyte is sofosbuvir. In some aspects, the analyte is rosuvastatin.

In other aspects, the analyte is a nucleic acid molecule, e.g., DNA, RNA, e.g., mRNA. In some aspects, the nucleic acid molecule is at least about 10 nucleic acids, at least about 15 nucleic acids, at least about 20 nucleic acids, at least about 25 nucleic acids, at least about 30 nucleic acids, at least about 40 nucleic acids, at least about 50 nucleic acids, at least about 100 nucleic acids, at least about 200 nucleic acids, at least about 300 nucleic acids, at least about 400 nucleic acids, at least about 500 nucleic acids, at least about 600 nucleic acids, at least about 700 nucleic acids, at least about 800 nucleic acids, at least about 900 nucleic acids, at least about 1000 nucleic acids, at least about 1200 nucleic acids, at least about 1400 nucleic acids, at least about 1600 nucleic acids, at least about 1800 nucleic acids, at least about 2000 nucleic acids, at least about 2200 nucleic acids, at least about 2400 nucleic acids, at least about 2600 nucleic acids, at least about 2800 nucleic acids, or at least about 3000 nucleic acids.

In some aspects, the analyte is an antisense oligonucleotide. In other aspects, the analyte is an siRNA or miRNA. In other aspects, the analyte is a gene therapy vector or a plasmid.

In some aspects, the SIL analyte contains at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20 isotope labels, at least about 21, at least about 22, at least about 23, at least about 24, at least about 25, at least about 26, at least about 27, at least about 28, at least about 29, at least about 30, at least about 31, at least about 32, at least about 33, at least about 34, at least about 35, at least about 36, at least about 37, at least about 38, at least about 39, or at least about 40 isotope labels.

Selection of the MIRM transitions for measurement is an important element of the present methods to ensure a robust and accurate assay performance. In some aspects, each of the measured relative peak area in MIRM transitions has less than 15% deviation from the calculated theoretical isotopic abundance in the corresponding MIRM transition of the SIL analyte or the naturally occurring isotopologues of the SIL analyte.

In some aspects, at least one of the measured relative peak area in MIRM transitions has less than 14%, less than 13%, less than 12%, less than 11%, less than 10%, less than 9%, less than 8%, less than 7%, less than 6%, less than 5%, less than 4%, less than 3%, less than 2%, less than 1%, less than 0.9%, less than 0.8%, less than 0.7%, less than 0.6%, less than 0.5%, less than 0.4%, less than 0.3%, less than 0.2%, less than 0.1%, less than 0.09%, less than 0.08%, less than 0.07%, less than 0.06%, less than 0.05%, less than 0.04%, less than 0.03%, less than 0.02%, less than 0.01%, less than 0.009%, less than 0.008%, less than 0.007%, less than 0.006%, less than 0.005%, less than 0.004%, less than 0.003%, less than 0.002%, less than 0.001%, less than 0.009%, less than 0.008%, less than 0.007%, less than 0.006%, less than 0.005%, less than 0.004%, less than 0.003%, less than 0.002%, or less than 0.0001% deviation from the calculated theoretical isotopic abundance in the corresponding MIRM transition of the SIL analyte or the naturally occurring isotopologues of the SIL analyte. In some aspects, all of the measured relative peak area in MIRM transitions has less than 14% deviation from the calculated theoretical isotopic abundance in the corresponding MIRM transition of the SIL analyte or the naturally occurring isotopologues of the SIL analyte. In some aspects, all of the measured relative peak area in MIRM transitions has less than 13% deviation from the calculated theoretical isotopic abundance in the corresponding MIRM transition of the SIL analyte or the naturally occurring isotopologues of the SIL analyte. In some aspects, all of the measured relative peak area in MIRM transitions has less than 12% deviation from the calculated theoretical isotopic abundance in the corresponding MIRM transition of the SIL analyte or the naturally occurring isotopologues of the SIL analyte. In some aspects, all of the measured relative peak area in MIRM transitions has less than 11% deviation from the calculated theoretical isotopic abundance in the corresponding MIRM transition of the SIL analyte or the naturally occurring isotopologues of the SIL analyte. In some aspects, all of the measured relative peak area in MIRM transitions has less than 10% deviation from the calculated theoretical isotopic abundance in the corresponding MIRM transition of the SIL analyte or the naturally occurring isotopologues of the SIL analyte. In some aspects, all of the measured relative peak area in MIRM transitions has less than 9% deviation from the calculated theoretical isotopic abundance in the corresponding MIRM transition of the SIL analyte or the naturally occurring isotopologues of the SIL analyte. In some aspects, all of the measured relative peak area in MIRM transitions has less than 8% deviation from the calculated theoretical isotopic abundance in the corresponding MIRM transition of the SIL analyte or the naturally occurring isotopologues of the SIL analyte. In some aspects, all of the measured relative peak area in MIRM transitions has less than 7% deviation from the calculated theoretical isotopic abundance in the corresponding MIRM transition of the SIL analyte or the naturally occurring isotopologues of the SIL analyte.

In some aspects, the number of the MIRM transitions is at least two, at least three, at least four, at least five, at least six, at least seven, at least 8, at least 9, at least 10, at least 11, at least 12, at least 13, at least 14, at least 15, at least 16, at least 17, at least 18, at least 19, or at least 20. In some aspects, the number of MIRM transitions is between 4 and 15, between 4 and 14, between 5 and 13, between 5 and 12, between 6 and 12, between 6 and 11, between 7 and 11, between 7 and 10, between 8 and 10, or between 8 and 9. In some aspects, the number of MIRM transitions is between 4 and 10, between 4 and 9, between 5 and 9, between 6 and 9, between 6 and 8, or between 7 and 8. In some aspects, the number of the MIRM transitions is 6. In some aspects, the number of the MIRM transitions is 7. In some aspects, the number of the MIRM transitions is 10. In some aspects, the number of the MIRM transitions is 15.

In some aspects, the analyte concentration equivalents of the highest MIRM channel and the lowest MIRM channel is at least about 10, at least about 100, at least about 200, at least about 300, at least about 400, at least about 500, at least about 600, at least about 700, at least about 800, at least about 900, at least about 1000, at least about 1100, at least about 1200, at least about 1300, at least about 1400, at least about 1500, at least about 1600, at least about 1700, at least about 1800, at least about 1900, at least about 2000, at least about 2100, at least about 2200, at least about 2300, at least about 2400, at least about 2500, at least about 2600, at least about 2700, at least about 2800, at least about 2900, at least about 3000, at least about 3100, at least about 3200, at least about 3300, at least about 3400, at least about 3500, at least about 3600, at least about 3700, at least about 3800, at least about 3900, at least about 4000, at least about 4100, at least about 4200, at least about 4300, at least about 4400, at least about 4500, at least about 4600, at least about 4700, at least about 4800, at least about 4900, at least about 5000, at least about 5100, at least about 5200, at least about 5300, at least about 5400, at least about 5500, at least about 5600, at least about 5700, at least about 5800, at least about 5900, or at least about 6000 fold difference.

In some aspects, the calculated theoretical isotopic abundance of two selected MIRM transitions at least 0.01% apart, at least 0.05% apart, at least 0.1% apart, at least 0.5% apart, are at least 1% apart, at least 1.5% apart, at least 2% apart, at least 2.5% apart, at least 3% apart, at least 3.5% apart, at least 4% apart, at least 4.5% apart, at least 5% apart, at least 5.5% apart, at least 6% apart, at least 6.5% apart, at least 7% apart, at least 7.5% apart, at least 8% apart, at least 8.5% apart, at least 9% apart, at least 9.5% apart, at least 10% apart, at least 20% apart, at least 30% apart, at least 40% apart, or at least about 50% apart.

In some aspects, the SIL analyte contains trace amounts of non-labeled analyte. In some aspects, the SIL analyte contains less than 5%, less than 4%, less than 3%, less than 2%, less than 1%, less than 0.9%, less than 0.8%, less than 0.7%, less than 0.6%, less than 0.5%, less than 0.4%, less than 0.3%, less than 0.2%, or less than 0.1% non-labeled analyte.

In some aspects, the SIL analyte is labeled at one or more positions with one or more stable isotopes. In some aspects, the stable isotope labels are ²H, ¹³C, ¹⁵N, ³³S, ³⁴S, ³⁶O, ¹⁷O, or ¹⁸O. In some aspects, the stable isotope labels are ¹³C and/or ¹⁵N. In some aspects, the stable isotope labels are ¹³C. In some aspects, the stable isotope labels are ¹⁵N.

Generally, for an experiment on a triple-quadrupole mass spectrometer, the first quadrupole (Q1) is set to pass ions only of a specified m/z (precursor ions) of an expected chemical species in the sample. The second quadrupole (i.e., Q2 or the collision cell) is used to fragment the ions passing through Q1. The third quadrupole (Q3) is set to pass to the detector only ions of a specified m/z (fragment ions) corresponding to an expected fragmentation product of the expected chemical species. In some aspects, the sample is ionized in the mass spectrometer to generate one or more protonated or deprotonated molecular ions. In some aspects, the one or more protonated or deprotonated molecular are singly charged, doubly charged, triply charged or higher. In some aspects, the mass spectrometer is a triple quadrupole mass spectrometer. In some aspects, the resolutions used for Q1 and Q3 are unit resolution. In other aspects, the resolutions used for Q1 and Q3 are different. In other aspects, the resolution used for Q1 is higher than the unit resolution of Q3.

The utility of separations by high performance liquid chromatography has been demonstrated over a broad range of applications including the analysis and purification of molecules ranging from low to high molecular weights. In liquid chromatography, there are significant limitations particularly arising out of the time required for analysis. The present methods are highly effective and improving the total required instrument time especially in the instance where an external calibration curve does not have to be run on the instrument. In some aspects, the method reduces a total instrument run time. In some aspects, an external calibration curve is not used. In some aspects, the analyte is a biomarker. In some aspects, the analyte is a metabolite.

The present methods are effective for detecting or quantifying analytes from a variety of sources, including biological sources. In some aspects, the sample is serum, tissue, biopsy tissue, formalin fixed paraffin embedded (FFPE), plasma, saliva, cerebral spinal fluid, tear, urine, synovial fluid, dried blood spot, or any combination thereof. In some aspects, the sample is serum. In some aspects, the sample is tissue. In some aspects, the sample is biopsy tissue. In some aspects, the sample is formalin fixed paraffin embedded (FFPE). In some aspects, the sample is plasma. In some aspects, the sample is saliva. In some aspects, the sample is cerebral spinal fluid. In some aspects, the sample is tear. In some aspects, the sample is urine. In some aspects, the sample is synovial fluid. In some aspects, the sample is dried blood spot.

The present methods are also useful to construct a liquid chromatography-mass spectrometry system comprising a liquid chromatography including at least one liquid chromatography column capable of separating an analyte from a biological matrix, a sample comprising the analyte of interest, at least one stable isotopically labeled analyte added to the sample, and a mass spectrometer capable of ionizing, fragmenting, and detecting one or more protonated or deprotonated parent ions and daughter ions specific to the analyte and the stable isotopically labeled analyte. Generally, chromatography is any kind of technique which separates a molecule (e.g., an antibody) from other molecules (e.g., contaminants) present in a mixture.

Liquid chromatography (LC) is a well-established analytical technique for separating components of a fluidic mixture for subsequent analysis and/or identification, in which a column, microfluidic chip-based channel, or tube is packed with a stationary phase material that typically is a finely divided solid or gel such as small particles with diameter of a few microns. The small particle size provides a large surface area that can be modified with various chemistries creating a stationary phase. A liquid eluent is pumped through the liquid chromatographic column (“LC column”) at a desired flow rate based on the column dimensions and particle size. This liquid eluent is sometimes referred to as the mobile phase. The sample to be analyzed is introduced (e.g., injected) in a small volume into the stream of the mobile phase prior to the LC column. The migration rates of analytes in the sample are affected by specific chemical and/or physical interactions with the stationary phase as they traverse the length of the column. The time at which a specific analyte elutes or comes out of the end of the column is called the retention time or elution time and can be a reasonably identifying characteristic of a given analyte especially when combined with other analyzing characteristics such as the accurate mass of a given analyte. The separated components may be passed from the liquid chromatographic column into other types of analytical instruments for further analysis, e.g., liquid chromatography-mass spectrometry (LC/MS or LC/MS/MS) separates compounds chromatographically before they are introduced to the ion source of a mass spectrometer.

Mass spectrometry (“MS” or “mass-spec”) is an analytical technique used to measure the mass-to-charge ratio ions. This is achieved by ionizing the sample and separating ions of differing masses and recording their relative abundance by measuring intensities of ion flux. A typical mass spectrometer comprises three parts: an ion source, a mass analyzer, and a detector system. The ion source is the part of the mass spectrometer that ionizes the substance under analysis (the analyte). The ions are then transported by magnetic or electric fields to the mass analyzer that separates the ions according to their mass-to-charge ratio (m/z). Many mass spectrometers use two or more mass analyzers for tandem mass spectrometry (MS/MS). The detector records the charge induced or current produced when an ion passes by or hits a surface. A mass spectrum is the result of measuring the signal produced in the detector when scanning m/z ions with a mass analyzer.

The present methods disclose a composition comprising an In-Sample Calibration Curve (ISCC) wherein ISCC comprises multiple isotopologue reaction monitoring (MIRM) of a stable isotopically labeled analyte. The present methods of generating an ISCC comprising MIRM of a stable isotopically labeled analyte are useful for the analysis of biomarkers. A biomarker can, for example, be isolated from the biological sample, directly measured in the biological sample, or detected in or determined to be in the biological sample. A biomarker can be functional, partially functional, or non-functional. If the biomarker is a protein or fragment thereof, it can be sequenced and its encoding gene can be cloned using well-established techniques. The present methods are also useful for the development and/or validation of a biomarker for regulatory acceptance as a surrogate endpoint. A surrogate endpoint is a biomarker accepted by regulatory agencies as a substitute for a clinical endpoint, and is intended to be used as a substitute for a clinically meaningful endpoint. Before a surrogate endpoint can be accepted, there must be extensive evidence showing that it can be relied upon to predict or correlate with clinical benefit.

The methods of the present disclosure are also useful for the analysis of biomarkers, metabolites or metabolic profiles. Analysis of biomarkers or metabolites represents a sensitive measure of biological status in health or disease. The altered metabolic fingerprints, which are unique to every individual, offer novel avenues to better understand systems biology, detect or identify potential risks for various diseases, and ultimately help achieve the goal of personalized medicine (i.e. the right drug(s), at the right dose, for the right person at the right time). A metabolite profile as used in the invention should be understood to be any defined set of values of quantitative results for metabolites that can be used for comparison to reference values or profiles derived from another sample or a group of samples. For instance, a metabolite profile of a sample from a diseased patient might be significantly different from a metabolite profile of a sample from a similarly matched healthy patient. A metabolite profile may aid in predicting a subject's susceptibility to a disorder by comparing the profile to a reference or standard profile.

The present methods also relate to recommending or selecting an optimal treatment protocol and/or an optimal drug selection, combination and dosage for a particular patient. Peak concentrations of a drug after each dose can be measured. The trough concentration of a drug after each dose can also be measured. The dosing interval (in time) including variation in that time, can be optimized based on information discerned from the analysis of biomarkers or metabolites.

The present disclosure also includes a liquid chromatography-mass spectrometry system used by the present methods described herein. In some aspects, the LC-MS/MS comprises:

(i) a liquid chromatography including at least one liquid chromatography column capable of separating an analyte from a biological matrix;

(ii) a sample comprising the analyte of interest;

(iii) at least one stable isotopically labeled analyte added to the sample; and

(iv) a mass spectrometer capable of ionizing, fragmenting, and detecting one or more protonated (or deprotonated) parent ions and daughter ions specific to the analyte and the stable isotopically labeled analyte.

In other aspects, the present disclosure includes a composition comprising an In-Sample Calibration Curve (ISCC) wherein ISCC comprises a stable isotopically labeled analyte.

The present disclosure is also directed to preparation of a one-sample multipoint external calibration curve (OSMECC). This approach does not use a stably labeled isotope (SIL) analyte and instead involved spiking a known amount of an analyte into a blank matrix sample. A blank matrix is a type of matrix does not contain the analyte of interest. By spiking a known amount of an analyte into one blank matrix sample, a one-sample multipoint external calibration curve in the blank matrix sample can be established on the basis of the relationship between the calculated theoretical isotopic abundances (analyte concentration equivalents) and the measured MS/MS peak areas (or peak area ratios if an internal standard is used for the assay) in the corresponding MIRM channels of the analyte. This one-sample multipoint external calibration curve can be used in the same way as the traditional multisample external calibration curve for quantitative LC-MS/MS-based bioanalysis. This approach serves as an alternate method to eliminate the need to prepare the traditional multisample external calibration curves in LC-MS/MS quantitative analysis.

As isotopic abundance in each MIRM channel can be calculated and measured accurately, isotope sample dilution can be achieved by simply monitoring one or a few of the MIRM channels of the analyte in addition to the most abundant MIRM channel for study samples. While the most abundant MIRM channel (isotopic abundance of 100%) is used for the quantitation of samples having concentrations within the assay calibration curve range, less abundant MIRM channels (isotopic abundance of IA %) can be used for the quantitation of samples having concentrations beyond the assay upper limit of quantitation (ULOQ), resulting in isotope dilution factors (IDF) of 100%/IA %. This approach serves as an alternate method to eliminate the need to physically dilute study samples in LC-MS/MS quantitative analysis.

In some aspects, the present disclosure is related to a method for quantifying the concentration of at least one analyte in a study sample, the method comprising adding one or more known amount(s) of one or more analyte(s) to a blank matrix sample to construct one or more One-Sample Multipoint External Calibration Curve(s) (OSMECC) by Multiple Isotopologue Reaction Monitoring (MIRM) of each added analyte(s), wherein the MIRM of an analyte refers to multiple reaction monitoring of multiple isotope transitions of the analyte; wherein the OSMECC for each analyte is constructed in the blank matrix sample based on the relationship between the calculated theoretical isotopic abundances (analyte concentration equivalents) in the MIRM transitions and the measured tandem mass spectrometry (MS/MS) peak areas (or peak area ratios if an internal standard is used for the assay) in the corresponding MIRM transitions; wherein the concentration of the at least one analyte in the study sample is quantified using the established OSMECC and the measured peak area (or peak area ratio if an internal standard is used for the assay) for the analyte from a liquid chromatography-tandem mass spectrometry (LC-MS/MS) process, wherein the peak area ratio for the analyte is the peak area of the analyte divided by the peak area of the internal standard, and wherein a tandem mass spectrometer is operated in multiple reaction monitoring mode.

The present disclosure is further illustrated by the following examples which should not be construed as further limiting. The contents of all references cited throughout this application are expressly incorporated herein by reference.

### EXAMPLES

### Example 1

### Preparation of the SIL Analyte for MIRM-ISCC-LC-MS/MS Analysis

Formic Acid (SupraPur grade) was purchased from EMD Chemicals (Gibbstown, N.J., USA). HPLC grade methanol and acetonitrile were purchased from J.T. Baker (Phillipsburg, N.J., USA). LC grade ammonium bicarbonate and phosphate buffered saline with 0.05% tween (PBST) were purchased from Sigma-Aldrich (St. Louis, Mo., USA). Dynabeads® M-280 Streptavidin was purchased from Invitrogen (Carlsbad, Calif., USA). Sequencing grade modified trypsin was purchased from Promega Corporation (Madison, Wis., USA). All non-labeled and labeled surrogate peptides for cluster of differentiation 73 (CD73): VIYPAVEGR (SEQ ID NO: 1) and V[Ile(¹³C₆, ¹⁵N)]YPAVEGR (SEQ ID NO: 1); programmed cell death protein 1 (PD-1): LAAFPEDR (SEQ ID NO: 2) and LAAFPED[Arg(¹³C₆, ¹⁵N₄)] (SEQ ID NO: 2); and programmed death-ligand 1 (PD-L1): LQDAGVYR (SEQ ID NO: 3) and LQDAG[Val(¹³C₅, ¹⁵N)]YR (SEQ ID NO: 3) were purchased from Genscript (Piscataway, N.J., USA). Deionized water was generated using a NANOpure Diamond ultrapure water system from Barnstead International (Dubuque, Iowa, USA). Recombinant human CD73 (61,084 Da), anti-human CD73 monoclonal antibody (mAb), small molecule drug daclatasvir and SIL drug, ¹³C₂¹⁵N₄-daclatasvir were generated.

The LC-MS/MS system used was a triple quadrupole 6500 mass spectrometer (AB Sciex, Foster City, Calif.) coupled with a Nexera UPLC system (Shimadzu, Columbia, Md.). The UPLC system consists of two LC-30AD pumps, one SIL-30ACMP autosampler and one CTO-30AS column heater. The separation was achieved on a Acquity HSS T3 analytical column (2.1 mm×50 mm, particle size 1.8 μm) (Waters, Milford, Mass.) with gradient elution using mobile phases of 0.01% formic acid in water (A) and 0.01% formic acid in acetonitrile (B). The LC-MS/MS data were acquired by Analyst® Software (1.6.2).

### Selection of MIRM Channels for Monitoring

FIG. 1 shows the general MIRM-ISCC-LC-MS/MS methodology using quantitative analysis of programmed death-ligand 1 (PD-L1) peptide LQDAGVYR (SEQ ID NO: 3) as an example. By spiking a known amount of a SIL analyte LQDAG[Val(¹³C₅, ¹⁵N)]YR (SEQ ID NO: 3) (20 μL of 500 ng/mL=10 ng) into each 100 μL study sample, the calculated theoretical isotopic abundances in the MIRM channels of this labeled analyte can be converted to the SIL analyte's isotope concentrations (or analyte concentration equivalents) in the corresponding MIRM channels. Therefore, an ISCC between the theoretical isotope concentrations (or analyte concentration equivalents) and the measured MS/MS responses can be established in each study sample, and the analyte concentration for this sample can be calculated instantly based on the established calibration curve and the analyte's peak area.

### MIRM-ISCC-LC-MS/MS Quantitation of Endogenous CD73 in Human and Money Serum

CD73 is a 70 kDa protein and has high expression on many tumors. Quantitative analysis of CD73 in human and monkey serum is needed to assist in dose selection and provide pharmacodynamic information for anti-CD73 pre-clinical and clinical drug development. Originally, an immuno-capture LC-MS/MS assay using traditional external calibration curves by serial dilution of a recombinant CD73 reference standard (61,084 Da) in surrogate matrix was developed and validated. An anti-CD73 mAb was used for immuno-capture of CD73, followed by denaturation, trypsin digestion, and LC-MS/MS analysis. The surrogate peptide (unique to both human and monkey CD73) monitored in the LC-MS/MS assay was VIYPAVEGR (SEQ ID NO: 1) with SRM transition (m/z) from a doubly charged parent ion to y6 ion (502.3⁺⁺→628.3⁺). A volume of 10 μL of the SIL surrogate peptide, V[Ile(¹³C₆, ¹⁵N)]YPAVEGR (SEQ ID NO: 1) at the concentration of 100 ng/mL was added into each sample after the trypsin digestion. As the original serum sample volume for this assay was 100 μL, this is equivalent to 10 ng/mL ([10 μL·100 ng/mL]/100 μL) of the SIL peptide in the original serum samples, or 604.792 ng/mL of recombinant CD73 (10 ng/mL·[61,084 Da/1010 Da]). In this experiment, this labeled peptide was used not only as the assay internal standard together with the external calibration curves, it was also used as an ISCC for each individual study sample as well. By doing this, the measured CD73 concentrations with the external calibration curves and ISCC can be compared side by side.

Before conducting the quantitation of CD73 in human and monkey serum using external calibration curve and ISCC approaches, the isotopic abundances in MIRM channels for the labeled surrogate peptide, V[Ile(¹³C₆, ¹⁵N)]YPAVEGR (SEQ ID NO: 1), need to be calculated and measured to confirm the selected MIRM channels are reliable and accurate to establish an ISCC without unexpected interferences.

The fragmentation of a peptide in triple quadrupole mass spectrometers can be easily resolved by using an online tool, such as Skyline (MacCoss Lab, Department of Genome Sciences, UW). In this case, as an y6 ion is monitored as the daughter ion, the daughter ion and neutral loss are determined to be C₂₆H₄₆N₉O₉⁺ and ¹³C₆¹⁵NC₁₄H₂₉N₂O₄, respectively. The isotopic distributions of the daughter ion (C₂₆H₄₆N₉O₉⁺) and neutral loss (¹³C₆¹⁵NC₁₄H₂₉N₂O₄) were calculated using an online calculator (worldwideweb.sisweb.com/mstools/isotope.html (accessed Nov. 10, 2019) and listed in Table 1. The isotopic abundances in the most abundant MIRM channel (100% abundance) and adjacent MIRM channels for V[Ile(¹³C₆, ¹⁵N)]YPAVEGR (SEQ ID NO: 1) were calculated. The calculated theoretical isotopic abundances in these MIRM channels and the measured results (peak areas) with a Sciex API 6500 mass spectrometer in the corresponding MIRM channels for V[ILE(¹³C₆, ¹⁵N)]YPAVEGR (SEQ ID NO: 1) are shown in Table 2. The abundances cover over 6,000-fold from the most to the least abundant MIRM channel. Lower isotopic abundances could also be calculated and used if necessary, however, in many cases, it will be beyond the linear range for a triple quadrupole mass spectrometer. As shown in Table 2, the percentage differences for the measured results from the calculated theoretical isotopic abundances are within 13.5%, indicating the measured results are accurate and reliable without any interferences, such as the interferences from isotope impurities and endogenous matrix, and therefore, these MIRM channels could be selected for MIRM-ISCC-LC-MS/MS absolute quantitative analysis.

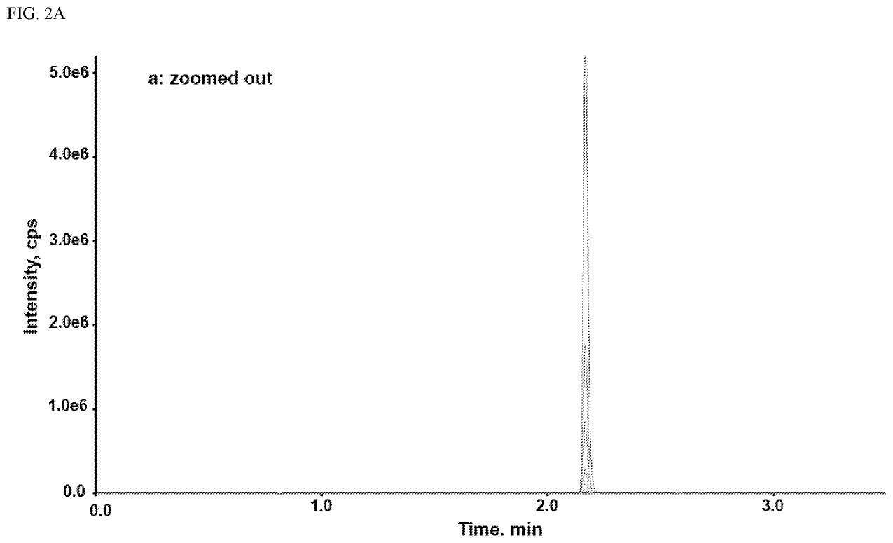

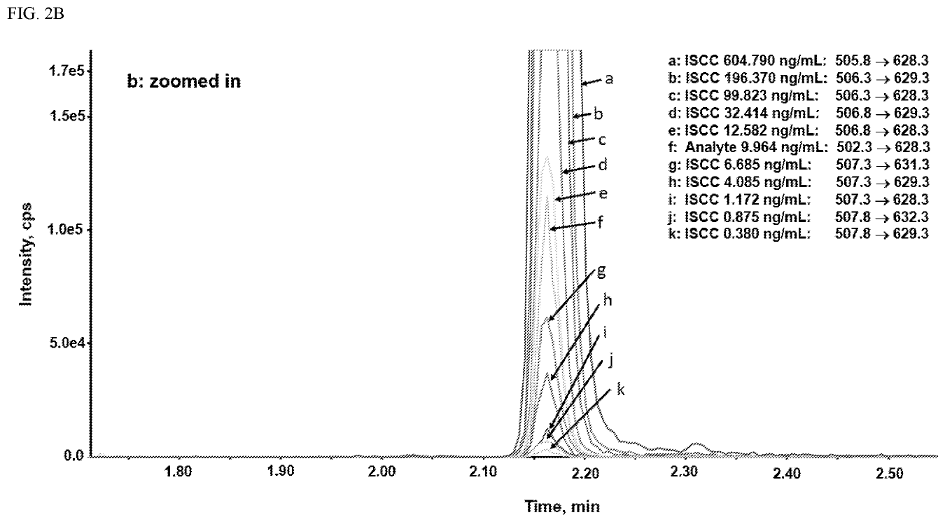

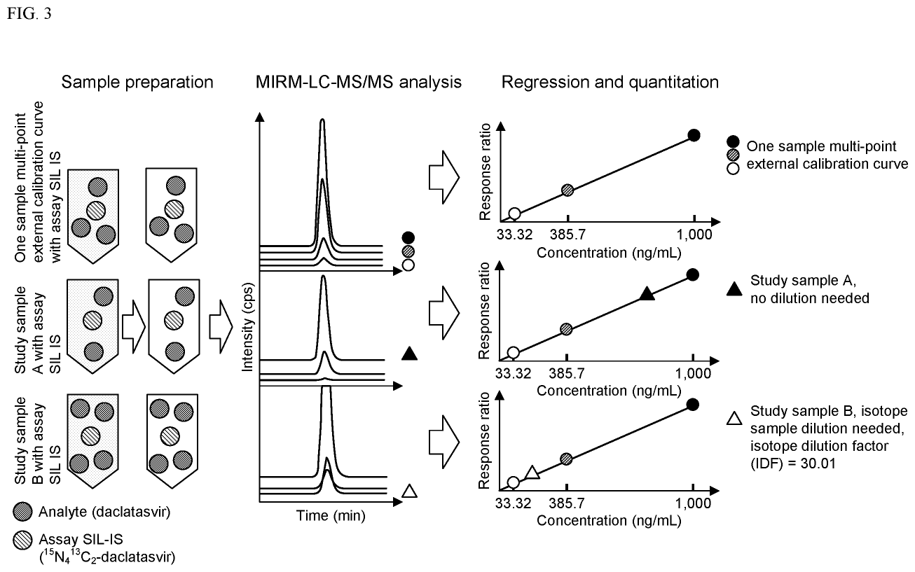

With the calculated theoretical isotopic abundances in the MIRM channels for the SIL peptide, the spiked SIL peptide isotope concentrations (and CD73 protein concentration equivalents) in the selected ten MIRM channels to be used for ISCC were calculated and listed in the right two columns in Table 2. An ISCC is constructed in each study sample using these concentrations (x axis) and the measured MS/MS peak areas (y axis) in the corresponding MIRM channels. Calibration curve regressions and concentration calculations were performed using an in-house developed software. A weighted (1/x²) least squares linear regression was used for all ISCCs. The ISCC performances, the linear curves (intercept and linear slope) and the calculated concentrations for the first three injections for sample No. 1 in human plasma (all samples were extracted and analyzed in three replicates) are shown in Table 3 and 4, respectively. As shown in Table 3, excellent ISCC performances were observed for these three replicates. Representative chromatograms for ten MIRM channels used for ISCC and one SRM channel for the analyte from the second injection are shown in FIGS. 2A and 2B.

The results for the quantitative analysis of endogenous CD73 levels in human and monkey serum using external calibration curve and ISCC approaches are listed in Table 5. Overall, CD73 concentrations measured with ISCC approach are about 11% to 17% lower than the concentrations measured using external calibration curve. This was caused by the 86.0% recovery for the immunocapture and digestion, as the immunocapture and digestion losses for the study samples were tracked and compensated by the external calibration curve. However, this was not the case for the ISCC approach as the SIL peptide was spiked after the digestion. Therefore, the concentrations measured with ISCC approach should be adjusted with the 86.0% recovery, and the adjusted concentrations matched with the concentrations measured using the external calibration curve very well, as shown in the Table 5.

It is not necessary to include all promising MIRM channels in the final assay. The selected MIRM channels for the quantitation of CD73 in human serum are noted in Table 2. There are several considerations in selecting MIRM channels to be used in sample analysis runs:

The ISCC curve range is defined by the isotopic abundance range of the selected MIRM channels. Therefore, appropriate MIRM channels should be selected to cover the expected concentration range. In this work, the selected MIRM channels covered about 1,600-fold curve range to cover the expected increase of CD73 after dose.

Any MIRM channel with large % Dev (>15%) between the calculated and measured isotopic abundances should not be selected as the large % Dev normally means that there is potential interference in the MIRM channel, including the interferences from isotope impurities and matrix endogenous. Therefore, multiple matrix lots should be tested for MIRM channel selection.

Only one MIRM channel should be selected among multiple MIRM channels with close isotopic abundances. In this example, as shown in Table 2, the MIRM channels 4 and 8 were not selected because the MIRM channels 4 and 5, 7 and 8 have very close isotopic abundances, respectively.

A total of ten MIRM channels were used in this example for demonstration purpose only. Using fewer MIRM channels (four to five MIRM channels for 1,000-fold curve) does not impact data quality.

TABLE 1
Isotopic distributions for neutral loss (¹³C₆¹⁵NC₁₄H₂₉N₂O₄)
and daughter ion ([C₂₆H₄₆N₉O₉]⁺) for stable isotopically labeled
peptide V[Ile(¹³C₆, ¹⁵N)]YPAVEGR (SEQ ID NO: 1)

| | Lost in collision cell | | | | |
|---|---|---|---|---|---|
| | (neutral loss) | | Mass | Daughter ion (y6 ion) | |
| Mass shift | ¹³C₆¹⁵NC₁₄H₂₉N₂O₄ | | shift for | [C₂₆H₄₆N₉O₉]⁺ | |
| for neutral | Mass | Abundance | daughter | | Abundance |
| loss: (α − β) | (m/z) | (%) | ion: β | Mass | (%) |
| 0 | 382.2 | 100 | 0 | 628.3 | 100 |
| 1 | 383.2 | 16.4891 | 1 | 629.3 | 32.4695 |
| 2 | 384.2 | 2.0781 | 2 | 630.3 | 6.9176 |
| 3 | 385.2 | 0.1935 | 3 | 631.3 | 1.1054 |
| 4 | 386.2 | 0.0145 | 4 | 632.3 | 0.1447 |
| 5 | 387.2 | 0.0008 | 5 | 633.3 | 0.0159 |
| | | | 6 | 634.3 | 0.0013 |

Note:
Parent ion: [¹³C₆¹⁵NC₄₀H₇₆N₁₁O₁₃]⁺⁺, doubly charged (Zₚ = 2)

TABLE 2
Calculated and measured relative isotopic abundances in MIRM channels of SIL
peptide V[ILE(¹³C₆, ¹⁵N)]YPAVEGR (SEQ ID NO: 1)

| | | Calculated | | | | Measured | | | | |
|---|---|---|---|---|---|---|---|---|---|---|
| | | theoretical | | | | relative | % Dev from | | Spiked ISCC | Spiked ISCC |
| | | relative | | | | isotopic | calculated | Selected | SIL peptide | CD73 protein |
| | MIRM | isotopic | Neutral | | Measured | abundance (%) | relative | MIRM | isotope | concentration |
| | channels | abundance | loss | Daughter | responses | based on peak | isotopic | channels for | concentration | equivalent |
| No | (m/z) | (%) | mass | ion mass | (peak area) | areas | abundance | ISCC | (ng/mL)^{a} | (ng/mL)^{b} |
| 1 | 505.8→6 | 100.0000 | 382.2 | 628.3 | 51529500 | 100.0000 | 0.0 | Yes | 10.0000 | 604.792 |
| | 28.3 | | | | | | | | | |
| 2 | 506.3→6 | 32.4695 | 382.2 | 629.3 | 18080800 | 35.0883 | 8.1 | Yes | 3.2470 | 196.376 |
| | 29.3 | | | | | | | | | |
| 3 | 506.3→6 | 16.4891 | 383.2 | 628.3 | 8961640 | 17.3913 | 5.5 | Yes | 1.6489 | 99.724 |
| | 28.3 | | | | | | | | | |
| 4 | 506.8→6 | 6.9176 | 382.2 | 630.3 | 3798610 | 7.3717 | 6.6 | No | | |
| | 30.3 | | | | | | | | | |
| 5 | 506.8→6 | 5.3539 | 383.2 | 629.3 | 2846230 | 5.5235 | 3.2 | Yes | 0.5354 | 32.381 |
| | 29.3 | | | | | | | | | |
| 6 | 506.8→6 | 2.0781 | 384.2 | 628.3 | 1077450 | 2.0909 | 0.6 | Yes | 0.2078 | 12.568 |
| | 28.3 | | | | | | | | | |
| 7 | 507.3→6 | 1.1054 | 382.2 | 631.3 | 618461 | 1.2002 | 8.6 | Yes | 0.1105 | 6.683 |
| | 31.3 | | | | | | | | | |
| 8 | 507.3→6 | 1.1406 | 383.2 | 630.3 | 657095 | 1.2752 | 11.8 | No | | |
| | 30.3 | | | | | | | | | |
| 9 | 507.3→6 | 0.6747 | 384.2 | 629.3 | 374215 | 0.7262 | 7.6 | Yes | 0.0675 | 4.082 |
| | 29.3 | | | | | | | | | |
| 10 | 507.3→6 | 0.1935 | 385.2 | 628.3 | 110815 | 0.2151 | 11.2 | Yes | 0.0194 | 1.173 |
| | 28.3 | | | | | | | | | |
| 11 | 507.8→6 | 0.1447 | 382.2 | 632.3 | 84638.5 | 0.1643 | 13.5 | Yes | 0.0145 | 0.877 |
| | 32.3 | | | | | | | | | |
| 12 | 507.8→6 | 0.1822 | 383.2 | 631.3 | 101662 | 0.1973 | 8.3 | No | | |
| | 31.3 | | | | | | | | | |
| 13 | 507.8→6 | 0.1438 | 384.2 | 630.3 | 77694.9 | 0.1508 | 4.9 | No | | |
| | 30.3 | | | | | | | | | |
| 14 | 507.8→6 | 0.0628 | 385.2 | 629.3 | 35250.9 | 0.0684 | 8.9 | Yes | 0.0063 | 0.381 |
| | 29.3 | | | | | | | | | |
| 15 | 507.8→6 | 0.0145 | 386.2 | 628.3 | 8305.59 | 0.0161 | 11.0 | No | | |
| | 28.3 | | | | | | | | | |
| | . . . | . . . | . . . | . . . | . . . | . . . | . . . | | | |

^{a}10 μL of 100 ng/mL (1 ng) of SIL peptide was spiked into the digested sample. As the original sample volume used for the assay was 100 μL, this is equivalent to that, in the original sample, there is 10 ng/mL of SIL peptide in its most abundant MIRM channel. Other SIL peptide isotope concentrations in adjacent MIRM channels were calculated based on the calculated theoretical relative isotopic abundances.
^{b}CD73 protein concentration equivalent = SIL peptide isotope concentration * (recombinant CD73 molecular weight of 61,084/SIL peptide molecular weight of 1010).

TABLE 3
Performances of ISCCs with 1/x² weighted linear regression for the first three injections

| ISCC nominal | 1st injection | | | 2nd injection | | | 3rd injection | | |
|---|---|---|---|---|---|---|---|---|---|
| concentration | | Predicted | | | Predicted | | | Predicted | |
| (ng/mL) | Peak area | concentration | % Dev | Peak area | concentration | % Dev | Peak area | concentration | % Dev |
| 604.792 | 12728343 | 593.664 | −1.8 | 12591027 | 611.535 | 1.1 | 13336529 | 609.439 | 0.8 |
| 196.376 | 4233869 | 197.466 | 0.6 | 4067840 | 197.559 | 0.6 | 4262965 | 194.766 | −0.8 |
| 99.724 | 2127881 | 99.239 | −0.5 | 1978622 | 96.084 | −3.6 | 2235632 | 102.115 | 2.4 |
| 32.381 | 696185 | 32.463 | 0.3 | 648848 | 31.497 | −2.7 | 720287 | 32.862 | 1.5 |
| 12.568 | 269720 | 12.571 | 0.0 | 246124 | 11.936 | −5.0 | 272520 | 12.398 | −1.4 |
| 6.683 | 145766 | 6.790 | 1.6 | 147531 | 7.147 | 6.9 | 149035 | 6.755 | 1.1 |
| 4.082 | 91382 | 4.253 | 4.2 | 85126 | 4.116 | 0.8 | 85889 | 3.869 | −5.2 |
| 1.173 | 25357 | 1.174 | 0.1 | 25394 | 1.215 | 3.6 | 29138 | 1.275 | 8.7 |
| 0.877 | 17703 | 0.817 | −6.9 | 18356 | 0.873 | −0.4 | 18833 | 0.805 | −8.3 |
| 0.381 | 8559 | 0.390 | 2.5 | 8122 | 0.376 | −1.3 | 9666 | 0.386 | 1.2 |

TABLE 4
ISCC linear curve intercepts, linear slopes and calculated
concentrations for the first three injections

| | | | Sample | Calculated |
|---|---|---|---|---|
| Injection | | Linear | peak | concentration |
| No. | Intercept | slope | area | (ng/mL) |
| 1 | 188.678 | 21439.995 | 168242 | 7.838 |
| 2 | 376.753 | 20588.607 | 205445 | 9.960 |
| 3 | 1229.704 | 21881.266 | 196858 | 8.940 |

Note:
Calculated concentration = (Sample peak area − Intercept)/Linear slope

TABLE 5
Results from quantitative analysis of endogenous CD73 in human
and monkey serum using external calibration curve and ISCC approaches

| | | | CD73 protein | | | Recovery adjusted | |
|---|---|---|---|---|---|---|---|
| | | | concentration (ng/mL) | CD73 protein | | CD73 protein | |
| Species/ | Sample | | using external | concentration (ng/mL) | | concentration (ng/mL) | |
| matrix | No. | Replicates | calibration curve | using ISCC | % Dev^{a} | using ISCC^{b} | % Dev^{c} |
| Human serum | 1 | 1 | 9.370 | 7.838 | −16.4 | 9.114 | −2.7 |
| | | 2 | 11.595 | 9.960 | −14.1 | 11.581 | −0.1 |
| | | 3 | 10.457 | 8.940 | −14.5 | 10.395 | −0.6 |
| | 2 | 1 | 4.412 | 3.740 | −15.2 | 4.349 | −1.4 |
| | | 2 | 4.047 | 3.453 | −14.7 | 4.015 | −0.8 |
| | | 3 | 5.005 | 4.216 | −15.8 | 4.902 | −2.1 |
| | 3 | 1 | 4.454 | 3.920 | −12.0 | 4.558 | 2.3 |
| | | 2 | 4.167 | 3.578 | −14.1 | 4.160 | −0.2 |
| | | 3 | 4.309 | 3.751 | −12.9 | 4.362 | 1.2 |
| Monkey Serum | 4 | 1 | 1.953 | 1.696 | −13.2 | 1.972 | 1.0 |
| | | 2 | 2.332 | 2.027 | −13.1 | 2.357 | 1.1 |
| | | 3 | 2.063 | 1.825 | −11.5 | 2.122 | 2.9 |
| | 5 | 1 | 2.773 | 2.333 | −15.9 | 2.713 | −2.2 |
| | | 2 | 2.811 | 2.429 | −13.6 | 2.824 | 0.5 |
| | | 3 | 2.786 | 2.471 | −11.3 | 2.873 | 3.1 |
| | 6 | 1 | 3.350 | 2.793 | −16.6 | 3.248 | −3.0 |
| | | 2 | 2.986 | 2.580 | −13.6 | 3.000 | 0.5 |
| | | 3 | 3.162 | 2.644 | −16.4 | 3.074 | −2.8 |

^{a}Percentage difference for the CD73 protein concentrations using ISCC from the CD73 protein concentrations using external calibration curve.
^{b}CD73 concentrations were adjusted with the recovery (86.0%) of immunocapture and digestion as the spiked SIL peptide did not go through the immunocapture and digestion steps.
^{c}Percentage difference for the recovery adjusted CD73 protein concentrations using ISCC from the CD73 protein concentration using external calibration curve.

### Example 2

### MIRM-ISCC-LC-MS/MS Quantitation of Surrogate Peptides for Protein Biomarkers PD-1, PD-L1 and CD73 in Digested Human Colon Homogenates

The MIRM-ISCC-LC-MS/MS methodology described above can be easily applied into quantitative proteomics by spiking known amounts of multiple SIL surrogate peptides into the digested samples for the absolute quantitative proteomics for multiple peptide targets. Similar to AQUA approach, the ISCC quantitation are also based on the concentrations of the SIL surrogate peptides spiked into the samples. However, as only one calibration point is used in AQUA approach for each target peptide, the accuracy of the quantitation could be greatly compromised, especially when the concentration for a target peptide is much higher or much lower than the concentration of the spiked SIL surrogate peptide. The MIRM-ISCC-LC-MS/MS approach, on the other hand, can offer a full calibration curve range with 3 to 4 orders of magnitude for each target peptide, and the accuracy of the quantitation can be assured within the entire curve range.

In this example, three surrogate peptides, LAAFPEDR (SEQ ID NO: 2) for PD-1, LQDAGVYR (SEQ ID NO: 3) for PD-L1 and VIYPAVEGR (SEQ ID NO: 1) for CD73, were mixed and spiked in fully trypsin digested human colon tissue homogenates at concentrations of 1.00, 10.0 and 50.0 ng/mL, respectively. A volume of 100 μL of the prepared sample was used in the assay. A mixture of 10 ng (20 μL of 500 ng/mL) for each SIL peptide LAAFPED[Arg(¹³C₆, ¹⁵N₄)] (SEQ ID NO: 2), LQDAG[Val(¹³C₅, ¹⁵N)]YR (SEQ ID NO: 3) and V[Ile(¹³C₆, ¹⁵N)]YPAVEGR (SEQ ID NO: 1) in 10% methanol 90% water was added into the prepared samples for MIRM-ISCC-LC-MS/MS analysis. The concentration for each of the SIL peptide in the samples is 100 ng/mL (10 ng/100 μL). Table 6 shows the MIRM channels, their isotope concentrations and analyte concentration equivalents used for MIRM-ISCC-LC-MS/MS quantitative analysis of these three peptides. A weighted (1/x²) least squares linear regression was used for all ISCC curves. Excellent ISCC curve performances were demonstrated by very accurate predicted concentrations for all calibration points (within 10.0% of the nominal concentrations, data not shown). The measured concentrations for these three peptides are listed in Table 7, and the accuracy of the MIRM-ISCC-LC-MS/MS measurement was confirmed by all of the samples tested.

TABLE 6
MIRM channels and their isotope concentrations used for MIRM-ISCC-LC-MS/MS
quantitative analysis of LAAFPEDR (SEQ ID NO: 2), LQDAGVYR (SEQ ID NO: 3) and
VIYPAVEGR (SEQ ID NO: 1) in digested human colon homogenates
LAAFPED[Arg(¹³C₆, ¹⁵N₄]⁺⁺ (SEQ ID NO: 2)→y4 ion⁺

| | | ISCC SIL- | |
|---|---|---|---|
| | | LAAFPEDR (SEQ | ISCC LAAFPEDR |
| | | ID NO: 2) isotope | (SEQ ID NO: 2) |
| | Isotopic abundance | concentration | concentration |
| MIRM channel (m/z) | (%) | (ng/mL) | equivalent (ng/mL)^{a} |
| 464.7→526.2 | 100 | 100 | 98.92 |
| 465.2→526.2 | 24.80 | 24.8 | 24.53 |
| 465.7→526.2 | 3.75 | 3.75 | 3.71 |
| 466.2→528.2 | 0.79 | 0.79 | 0.78 |
| 466.2→526.2 | 0.42 | 0.42 | 0.42 |

| | |
|---|---|
| 459.7→516.2 | SRM channel for analyte, LAAFPEDR (SEQ ID NO: 2) |

LQDAG[Val(_{¹³}C₅, _{¹⁵}N]YR_{⁺⁺} (SEQ ID NO: 3)→y6 ion_{⁺}

| | | ISCC SIL- | |
|---|---|---|---|
| | | LQDAGVYR (SEQ | ISCC LQDAGVYR |
| | | ID NO: 3) isotope | (SEQ ID NO: 3) |
| MIRM channel | Isotopic abundance | concentration | concentration |
| (m/z) | (%) | (ng/mL) | equivalent (ng/mL)^{b} |
| 464.2→686.4 | 100 | 100 | 99.35 |
| 464.7→687.4 | 29.99 | 30.0 | 29.81 |
| 465.2→688.4 | 6.63 | 6.63 | 6.59 |
| 465.7→689.4 | 1.01 | 1.01 | 1.00 |
| 465.7→687.4 | 0.43 | 0.43 | 0.43 |

| | |
|---|---|
| 461.2→680.4 | SRM channel for analyte, LQDAGVYR (SEQ ID NO: 3) |

V[Ile(¹³C₆, ¹⁵N]YPAVEGR⁺⁺ (SEQ ID NO: 1)→y6 ion⁺

| | | | ISCC VIYPAVEGR |
|---|---|---|---|
| | | ISCC SIL-VIYPAVEGR | (SEQ ID NO: 1) |
| MIRM channel | Isotopic | (SEQ ID NO: 1) isotope | concentration |
| (m/z) | abundance (%) | concentration (ng/mL) | equivalent (ng/mL)^{c} |
| 505.8→628.3 | 100 | 100 | 99.31 |
| 506.3→629.3 | 32.47 | 32.5 | 32.27 |
| 506.8→630.3 | 6.92 | 6.92 | 6.87 |
| 507.3→630.3 | 1.14 | 1.14 | 1.13 |
| 507.8→632.3 | 0.14 | 0.14 | 0.14 |

| | |
|---|---|
| 502.3→628.3 | SRM channel for analyte, VIYPAVEGR (SEQ ID NO: 1) |

^{a}ISCC LAAFPEDR (SEQ ID NO: 2) concentration equivalent = ISCC SIL-LAAFPEDR (SEQ ID NO: 2) isotope concentration * (LAAFPEDR (SEQ ID NO: 2) molecular weight of 918/SIL-LAAFPEDR (SEQ ID NO: 2) molecular weight of 928)
^{b}ISCC LQDAGVYR (SEQ ID NO: 3) concentration equivalent = ISCC SIL-LQDAGVYR (SEQ ID NO: 3) isotope concentration * (LQDAGVYR (SEQ ID NO: 3) molecular weight of 921/SIL-LQDAGVYR (SEQ ID NO: 3) molecular weight of 927)
^{c}ISCC VIYPAVEGR (SEQ ID NO: 1) concentration equivalent = ISCC SIL-VIYPAVEGR (SEQ ID NO: 1) isotope concentration * (VIYPAVEGR (SEQ ID NO: 1) molecular weight of 1003/SIL-VIYPAVEGR (SEQ ID NO: 1) molecular weight of 1010)

TABLE 7
Quantitative analysis of surrogate peptides LAAFPEDR (SEQ ID NO: 2),
LQDAGVYR (SEQ ID NO: 3) and VIYPAVEGR (SEQ ID NO: 1) in fully digested colon
tissue homogenates using MIRM-ISCC-LC-MS/MS

| | LAAFPEDR (SEQ ID | | LQDAGVYR | | VIYPAVEGR (SEQ ID | |
|---|---|---|---|---|---|---|
| Analyte | NO: 2) | | (SEQ ID NO: 3) | | NO: 1) | |
| Nominal | Measured | | Measured | | Measured | |
| concentration | concentration | | concentration | | concentration | |
| (ng/mL) | (ng/mL) | % Dev | (ng/mL) | % Dev | (ng/mL) | % Dev |
| Digested tissue | <0.42 | | <0.43 | | <0.14 | |
| homogenate blank | <0.42 | | <0.43 | | <0.14 | |
| | <0.42 | | <0.43 | | <0.14 | |
| 1.00 | 1.03 | 3.0 | 0.95 | −5.0 | 1.17 | 17.0 |
| | 1.06 | 6.0 | 1.02 | 2.0 | 1.15 | 15.0 |
| | 1.03 | 3.0 | 0.99 | −1.0 | 1.20 | 20.0 |
| 10.0 | 9.75 | −2.5 | 9.59 | −4.1 | 10.92 | 9.2 |
| | 10.09 | 0.9 | 10.03 | 0.3 | 10.92 | 9.2 |
| | 9.84 | −1.6 | 9.86 | −1.4 | 10.92 | 9.2 |
| 50.0 | 49.86 | −0.3 | 48.48 | −3.0 | 53.33 | 6.7 |
| | 47.78 | −4.4 | 48.88 | −2.2 | 52.24 | 4.5 |
| | 49.76 | −0.5 | 48.29 | −3.4 | 52.83 | 5.7 |

One potential issue for MIRM-ISCC-LC-MS/MS approach in targeted quantitative proteomics for multiple peptides is that the total number of MIRM channels needs to be monitored in a LC-MS/MS run could be too many to be handled by a triple quadrupole mass spectrometer. This issue could be relieved by using scheduled MIRM based on the different retention time windows for each target peptide, and using fewer MIRM channels in each ISCC. Results indicated that accurate and reliable quantitation still could be achieved by using 3 to 4 MIRM channels for a concentration range of 2 to 3 orders of magnitude in a multiplexed fashion.

### Example 3

### MIRM-ICSS-LC-MS/MS Analysis of Small Molecule Drug Daclatasvir in Human and Rat Plasma

After the daughter ions and neutral losses are determined, the same MIRM-ISCC-LC-MS/MS work flow can also be used for the measurement of small molecule analytes, including small molecule drugs and biomarkers. Here we show an example of instant quantitative analysis of a small molecule drug, daclatasvir, in human and rat plasma using MIRM-ISCC-LC-MS/MS approach.

100 μL of human and rat plasma samples at 10, 100, 500 and 1,000 ng/mL of daclatasvir were mixed with 20 μL of 5,000 ng/mL of SIL ¹³C₂¹⁵N₄-daclatasvir in human, and rat plasma, respectively. The equivalent concentration of ¹³C₂¹⁵N₄-daclatasvir in human and rat plasma samples was 1,000 ng/mL ([20 μL×5,000 ng/mL]/100 μL=1,000 ng/mL). The samples were extracted with liquid-liquid extraction (H. Jiang et al, Journal of Chromatography A 2012, 1245, 117-121) and injected for MIRM-ISCC-LC-MS/MS analysis. Table 8 shows the MIRM channels and their isotope concentrations used in ISCC for quantitation of daclatasvir. All ISCCs were constructed using a weighted (1/x²) least squares linear regression. The predicted concentrations for all calibration points are well within the acceptance criteria for regulated LC-MS/MS bioanalysis (data not shown). Table 9 shows the measured results for daclatasvir in human and rat plasma, indicating the MIRM-ISCC-LC-MS/MS analysis of daclatasvir was accurate.

TABLE 8
MIRM channels and their isotope concentrations used for
MIRM-ISCC-LC-MS/MS quantitative analysis of daclatasvir

| | | ISCC SIL- | ISCC |
|---|---|---|---|
| | | daclatasvir | daclatasvir |
| MIRM channel (m/z) | Isotopic | isotope | concentration |
| ¹³C₂¹⁵N₄C₃₈H₅₁N₄O₆⁺ → | abundance | concentration | equivalent |
| ¹³C₂¹⁵N₄C₃₁H₃₇N₂O₃⁺ | (%) | (ng/mL) | (ng/mL)^{a} |
| 745.4 → 571.3 | 100 | 1,000 | 991.9 |
| 746.4 → 572.3 | 34.96 | 349.6 | 346.8 |
| 746.4 → 571.3 | 8.64 | 86.4 | 85.7 |
| 747.4 → 572.3 | 3.02 | 30.2 | 30.0 |
| 747.4 → 571.3 | 0.93 | 9.30 | 9.23 |
| 748.4 → 573.3 | 0.56 | 5.60 | 5.55 |

| | |
|---|---|
| 739.4 → 565.3 | SRM channel for analyte, daclatasvir |

^{a}ISCC daclatasvir concentration equivalent = ISCC SIL-daclatasvir isotope concentration * (daclatasvir molecular weight of 739/SIL-daclatasvir molecular weight of 745)

TABLE 9
Quantitative analysis of daclatasvir in human
and rat plasma using MIRM-ISCC-LC-MS/MS

| | Human plasma | | Rat plasma | |
|---|---|---|---|---|
| Nominal | Measured | | Measured | |
| concentration | concentration | | concentration | |
| (ng/mL) | (ng/mL) | % Dev | (ng/mL) | % Dev |
| 10.00 | 10.81 | 8.1 | 12.72 | 27.2 |
| | 11.69 | 16.9 | 10.72 | 7.2 |
| 100.0 | 107.7 | 7.7 | 110.2 | 10.2 |
| | 110.1 | 10.1 | 111.8 | 11.8 |
| 500.0 | 523.9 | 4.8 | 574.2 | 14.8 |
| | 550.7 | 10.1 | 567.4 | 13.5 |
| 1,000 | 1066 | 6.6 | 1045 | 4.5 |
| | 1087 | 8.7 | 1077 | 7.7 |

### Additional Considerations for MIRM-ISCC-LC-MS/MS Assays

There are several additional considerations for the successful MIRM-ISCC-LC-MS/MS assay development and sample analysis. Selection of a proper SIL analyte is one of the important factors to develop a reliable and robust quantitative MIRM-ISCC-LC-MS/MS assay. As the isotopic abundances of this labeled analyte in the selected MIRM channels will be used as a calibration curve for quantitative analysis of the analyte, the labeled analyte should be designed to avoid the isotopic interference from the analyte as this interference could compromise the assay accuracy. The rule of thumb is that four to six labels are needed to avoid the interference for most small molecule compounds and peptide analytes with 6 to 12 amino acids. The impurity (amount of non-labeled analyte) in the SIL analyte should be low enough to avoid the interference from the SIL analyte to the analyte because, for ISCC approach, a large amount of the SIL analyte is needed in each study sample to define the assay upper limit of quantitation (ULOQ). In addition, the labeling impurity (amount of labeled analyte with fewer or more labeled positions than that of the SIL analyte) should also be low enough to avoid the interferences to the isotopic abundances in the MIRM channels of the SIL analyte.

Although deuterium labeling is very cost effective and easily available, deuterium labeled analytes should be avoided in the ISCC approach due to the easy separation of the deuterium labeled analytes from the non-labeled analytes, and more importantly, the hydrogen-deuterium exchange reaction, which can easily occur on exchangeable protons and deuterons, makes the accurate calculation of the isotopic abundances in MIRM channels impossible.

Using properly calibrated triple quadrupole mass spectrometer is another factor for the success of MIRM-ISCC-LC-MS/MS approach. For singly- and doubly-charged parent ions, unit resolutions (full width at half height—FWHH=0.7 mass unit) for both Q1 and Q3 are good enough to generate accurate MS/MS responses close to the calculated theoretical isotopic abundances in the corresponding MIRM channels. If necessary, higher resolution (FWHH=0.5 mass unit) in Q1 can improve the measurement accuracy, with the cost of losing some instrument sensitivity. Our test results showed that using higher resolution (FWHH=0.5 mass unit) in Q3 is not helpful in improving the measurement accuracy.

As the isotope spacing (1 Da for Zp=1, 0.5 Da for Zp=2, 0.33 Da for Zp=3, and so on) gradually decreases with the increase of the number of charge (Zp), it is anticipated that accurate measurement of isotopic abundances in MIRM channels with triply (and higher) charged parent ions might be challenging even using resolutions with FWHH≤0.5 mass unit. In this work, isotopic abundances in MIRM channels with triply (or higher) charged parent ions were not tested because triple (or higher) charged parent ions with high MS/MS responses were not available.

The performances for an established ISCC should be very similar from sample to sample during the assay qualification and sample analysis as the ISCC is constructed using naturally occurring isotopic abundances in selected MIRM channels, with no human and instrument operations involved. Any unexpected significant bias in one MIRM channel for a few samples normally indicates endogenous interferences from those matrix lots, and excluding this point has no significant impact on the data accuracy. On the other hand, any unexpected significant biases in most MIRM channels for many samples indicate failure of MS instrument calibration.

There is no need to add an additional assay internal standard in the MIRM-ISCC-LC-MS/MS approach because an ISCC is in each study sample, and therefore all variations after the spiking of a SIL analyte into the study samples, including variations from extraction, injection, ionization, fragmentation and detection, etc., are tracked and compensated by the ISCC itself. Because of this, the assay performance could be further improved by spiking the SIL analyte as early as possible during the sample preparation, such as the analysis of small molecule drug daclatasvir in the example 3 where the SIL daclatasvir was spiked into the samples at the beginning of the sample preparation. However, for protein analysis with immuno-capture, the labeled peptides can only be spiked after immuno-capture, and any variations during immuno-capture and trypsin digestion are not tracked and compensated, such as the analysis of CD73 protein in the example 1. This issue can be resolved by spiking a SIL protein with the surrogate peptide portion labeled at the beginning of the sample preparation.

As an ISCC is in each individual sample and currently there is no commercial software which is capable to build ISCCs using peak areas from multiple MIRM channels, an in-house developed software was used in this work for generating ISCCs with weighted least squares regression algorithm, and calculating sample concentrations in batch. Wide application of MIRM-ISCC-LC-MS/MS methodology is relied on the commercial software development by major mass spectrometer companies.

### Example 4

Quantitative LC-MS/MS Bioanalysis with One-Sample Multipoint External Calibration Curve and Isotope Sample Dilution using MIRM Technique

The LC-MS/MS system used was a triple-quadrupole 6500 mass spectrometer (AB Sciex, Foster City, Calif.) coupled with a Nexera UPLC system (Shimadzu, Columbia, Md.). The UPLC system consists of two LC-30AD pumps, one SIL-30ACMP autosampler, and one CTO-30AS column heater. The separation was achieved on a Acquity HSS T3 analytical column (2.1 mm×50 mm, particle size 1.8 m) (Waters, Milford, Mass.) with gradient elution using mobile phases of 10 mM ammonium acetate in water/acetonitrile (90/10) containing 0.1% formic acid and 10 mM ammonium acetate in water/acetonitrile (10/90) containing 0.1% formic acid. The flow rate was 0.8 mL/min. The LC-MS/MS data were acquired by Analyst Software (1.6.2) (AB Sciex, Foster City, Calif.).

Sample Preparation for LC-MS/MS Quantitation of Daclatasvir Using Multisample External Calibration Curve, One-Sample Multipoint External Calibration Curve, and ISCCs. All stock solutions for daclatasvir and ¹³C₂¹⁵N₄-daclatasvir were prepared in acetonitrile/DMSO (1/1, v/v). Daclatasvir at 20000 ng/mL was prepared by appropriate dilution of the 0.5 mg/mL stock solution with human plasma. A multisample external calibration curve at concentrations of 1000, 800, 500, 100, 20, 4, 2, and 1 ng/mL for daclatasvir were prepared by serial dilution from 20000 ng/mL of daclatasvir in human plasma. QC samples at 20000, 5000, 800, 500, 40, 3, and 1 ng/mL for daclatasvir were prepared by serial dilution from the 0.5 mg/mL daclatasvir stock solution.

Daclatasvir at a concentration of 5000 ng/mL was prepared in methanol/water (10/90) by appropriate dilution of the 0.5 mg/mL of daclatasvir stock solution, and this solution was used to build a one-sample multipoint external calibration curve. 13C₂¹⁵N₄-Daclatasvir at 5040.6 ng/mL was prepared by appropriate dilution of the 0.5 mg/mL ¹³C₂¹⁵N₄-daclatasvir stock solution by methanol/water (10/90), and this solution was used to build ISCCs in each sample and was also used as the assay stable isotopically labeled internal standard (SIL-IS) for both multisample external calibration curve and one-sample multipoint external calibration curve approaches.

An aliquot of 100 μL of plasma samples for two multisample external calibration curves from 1 to 1000 ng/mL, six replicates of QC samples at 1, 3, 40, 500, and 800 ng/mL, and two one-sample multipoint external calibration curves (two blank plasma samples to be spiked with a known amount of daclatasvir) were transferred into a 96-well plate. QC samples at 5000 and 20000 ng/mL were diluted 100- and 200-fold, respectively, and 100 μL of both diluted and non-diluted QC samples at 5000 and 20000 ng/mL (6 replicates each) were transferred into the 96-well plate. A volume of 20 μL of 5000 ng/mL of daclatasvir in 10% methanol and 90% water was added into the two blank human plasma samples to construct two one-sample multipoint external calibration curves, and a volume of 20 μL of 10% methanol and 90% water was added into other samples as a makeup. An aliquot of 20 μL of 5040.6 ng/mL ¹³C₂¹⁵N₄-daclatasvir in 10% methanol and 90% water was added into each plasma samples in the 96-well plate. ¹³C₂¹⁵N₄-Daclatasvir was used as the assay SIL-IS for the multisample external calibration curves and one-sample multipoint external calibration curves. It was also used as an SIL analyte to construct ISCCs for each sample.

The samples were extracted with liquid-liquid extraction (H. Jiang et al, Journal of Chromatography A 2012, 1245, 117-121) using a Janus Mini liquid handler (PerkinElmer, Waltham, Mass.). A volume of 50 μL of 1 M ammonium bicarbonate buffer was added to each sample followed by 600 μL of MTBE. The 96-well plate was vortexed for 5 min, and 400 μL of supernatant was transferred to a clean 96-well plate and evaporated to dryness at 50° C. The samples were reconstituted with 100 μL of 10% methanol in water for LC-MS/MS analysis.

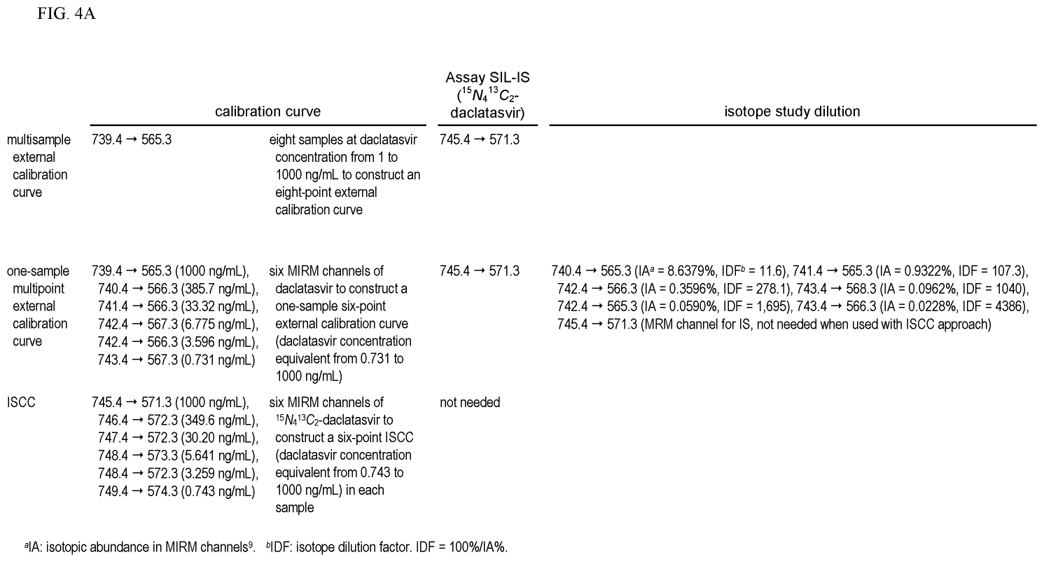

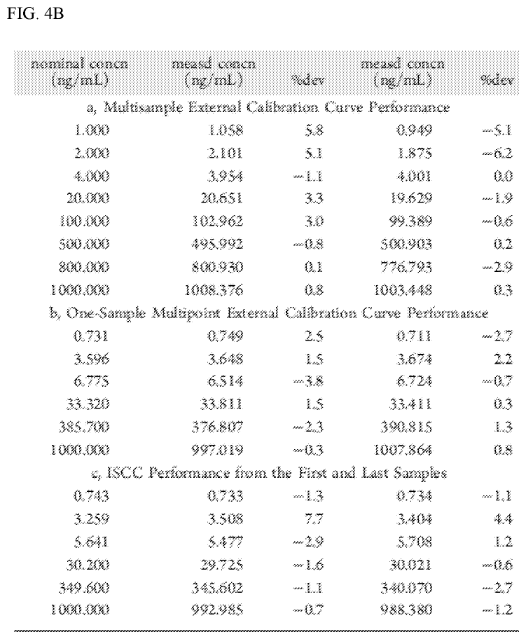

A summary of the MRM and MIRM transitions monitored for each of the multisample external calibration curves, one-sample multipoint external calibration curve (OSMECC) and the in-sample calibration curve (ISCC) are shown in FIG. 4A. The SRM channels used for isotope sample dilution and the corresponding isotope dilution factor (IDF) are also shown in FIG. 4A. The calibration curve performances for two multisample external calibration curves and two one-sample multipoint external calibration curves used, as well as two ISCCs, are shown in FIG. 4B.

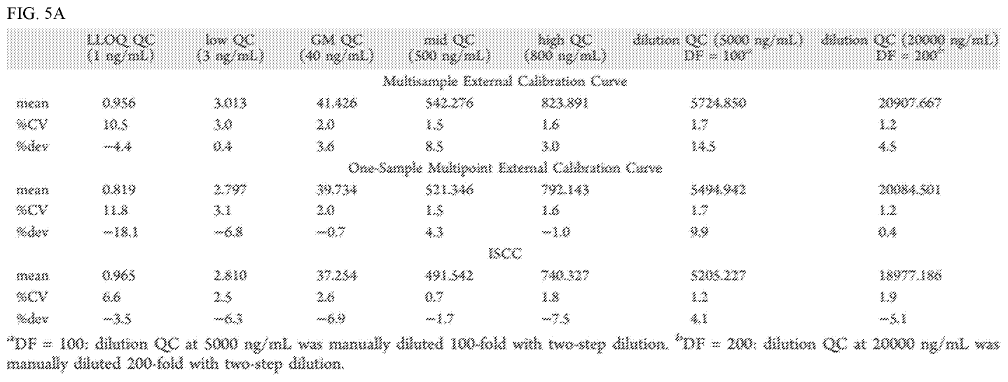

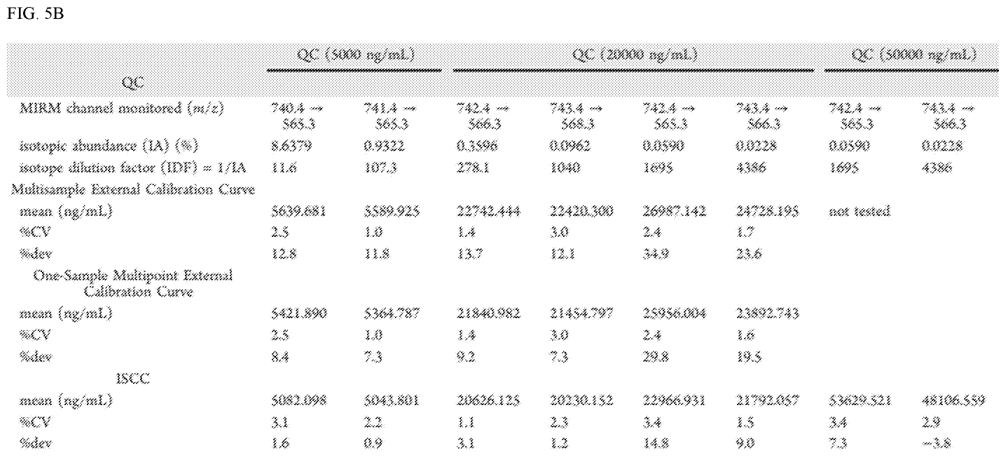

The accuracy and precision data for QC samples using multisample external calibration curves, one-sample multipoint external calibration curves, and ISCCs are shown in FIG. 5A. QC samples at 5000 and 20000 ng/mL were physically diluted 100-fold and 200-fold with two-step dilution, respectively. In this example, the concentrations of the same set of QC samples were calculated with the three different types of calibration curves. The accurate measurements for the QC samples at all concentration levels with these three type of calibration curves demonstrated that both one-sample multipoint calibration curves and ISCCs can deliver bioanalytical data with the same level of accuracy as the traditional multisample external calibration curves. Therefore, they can be used in LC-MS/MS bioanalytical assays where multisample external calibration curves are traditionally used.

To eliminate the sample dilution step for the samples with concentrations above the assay's ULOQ, the isotope sample dilution approach using the MIRM technique was evaluated by using QC samples at 5000, 20000 and 50000 ng/mL with all three types of calibration curves. As shown in FIG. 5B, accurate measurements for QC samples at 5000 and 20000 ng/mL were achieved using three different calibration curves with isotope dilution factor (IDF) up to 1040-fold. Only ISCC provided accurate measurement for the QC sample at 20000 ng/mL with IDFs of 1695 and 4386. Additionally, IDFs of 1695 and 4386 were evaluated for ISCC by using QC samples at concentration of 50000 ng/mL, and accurate measurements were achieved for both IDFs. Therefore, the isotope sample dilution approach can be used in LC-MS/MS bioanalytical analysis to eliminate the physical sample dilution step.

Throughout this application, various publications are referenced in parentheses by author name and date, or by patent No. or Patent Publication No. The disclosures of these publications are hereby incorporated in their entireties by reference into this application in order to more fully describe the state of the art as known to those skilled therein as of the date of the disclosure described and claimed herein. However, the citation of a reference herein should not be construed as an acknowledgement that such reference is prior art to the present disclosure.

What is claimed is:

1. A method for quantifying the concentration of at least one analyte in a sample, the method comprising adding one or more known amount(s) stable isotopically labeled (SIL) analyte(s) to a sample containing at least one analyte to construct one or more In-Sample Calibration Curve(s) (ISCC) by Multiple Isotopologue Reaction Monitoring (MIRM) of each added SIL analyte(s), wherein the MIRM of an SIL analyte refers to multiple reaction monitoring of multiple isotope transitions of the SIL analyte; wherein the ISCC for each analyte is constructed in the sample based on the relationship between the calculated theoretical isotopic abundances (analyte concentration equivalents) in the MIRM transitions and the measured tandem mass spectrometry (MS/MS) peak areas in the corresponding MIRM transitions; wherein the concentration of the at least one analyte in the sample is quantified using the established ISCC and the measured peak area for the analyte from a liquid chromatography-tandem mass spectrometry (LC-MS/MS) process, and wherein a tandem mass spectrometer is operated in multiple reaction monitoring mode.

2. The method of claim 1, wherein
(i) the analyte, the SIL analyte and the naturally occurring isotopologues of the SIL analyte are ionized in the mass spectrometer to produce protonated (or deprotonated) parent ions of the analyte, the SIL analyte and the naturally occurring isotopologues of the SIL analyte;
(ii) the parent ions of the analyte, the parent ions of the SIL analyte and the parent ions of the naturally occurring isotopologues of the SIL analyte in the mass spectrometer are fragmented at the same cleavage site to produce neutral losses and daughter ions;
(iii) the transition from the parent ion to the daughter ion for the analyte is monitored in the mass spectrometer;
(iv) a peak area for the transition from the parent ion to the daughter ion for the analyte is measured;
(v) the selected multiple transitions from parent ions of the SIL analyte and the parent ions of the naturally occurring isotopologues of the SIL analyte to the daughter ions of the SIL analyte and the daughter ions of the naturally occurring isotopologues of the SIL analyte are monitored in the mass spectrometer (“multiple isotopologue reaction monitoring” or “MIRM”);
(vi) a peak area of each of the MIRM transitions is measured, wherein the MIRM transitions comprise the selected transitions from parent ions of the SIL analyte and the parent ions of the naturally occurring isotopologues of the SIL analyte to the daughter ions of the SIL analyte and the daughter ions of the naturally occurring isotopologues of the SIL analyte;

3. The method of claim 2, further generating an In-Sample Calibration Curve based on the relationship between the measured peak areas in the MIRM transitions of the SIL analyte and the naturally occurring isotopologues of the SIL analyte, and the analyte concentration equivalents for each of the MIRM transitions.

4. The method of claim 3, wherein the analyte concentration equivalent for each MIRM transition is calculated from a theoretical isotopic abundance of the corresponding MIRM transition of the SIL analyte or the naturally occurring isotopologues of the SIL analyte, wherein the theoretical isotopic abundance is calculated using a methodology published on Analytical Chemistry, 2012, 84(11), 4844-4850, wherein the methodology is calculated based on the isotope distributions of the neutral loss and the daughter ion of the SIL analyte.

5. The method of claim 4, wherein the theoretical isotopic abundance for each of the MIRM transition (m/z) from (p+Zₚ+α)/Zₚ to (d+Zd+β)/Z_{d} of the SIL analyte and the naturally occurring isotopologues of the SIL analyte is calculated based on formula (I):
Isotopic abundance in an MIRM transition of (p+Zₚ+α)/Zₚ→(d+Z_{d}+β)/Z_{d}=[relative isotope distribution of the daughter ion at mass of (d+Z_{d}+β)]*[relative isotope distribution of the neutral loss at mass of n+(α−β)] (I)
Wherein m/z is the mass to charge ratio
p is the monoisotopic mass of the parent molecule of the SIL analyte
Zₚ is the number of charge for the parent ion
d is the monoisotopic mass of the daughter fragment of the SIL analyte
Zd is the number of charge for the daughter ion
n is the monoisotopic mass of the neutral loss of the SIL analyte
p=d+n
α and β are integer, they are the number of additional neutrons on the parent ion and daughter ion, respectively, α≥0, β≥0 and α≥β
Zₚ and Z_{d} are integers

6. The method of claim 4 and 5, wherein the isotope distribution calculator is at worldwideweb.sisweb.com/mstools/isotope.html (accessed Nov. 10, 2019).

7. The method of claim 6, wherein the highest analyte concentration equivalent (“Upper Limit of Quantification” or “ULOQ” of the ISCC) is calculated based on formula (II):
(M/V)*(M_{analyte}/M_{SIL analyte}) ng/mL (II)
Wherein M (ng) is the total amount of the SIL analyte added into the sample;
V is the sample volume (mL) before the SIL analyte is added;
M_{analyte} is the molecular weight of the analyte;
M_{SIL analyte} is the molecular weight of the SIL analyte.

8. The method of claim 7, wherein one or more of the other analyte concentration equivalents in the MIRM transitions are calculated based on formula (III):
Iₐ*ULOQ (ng/ml) (III)
Wherein Iₐ is the calculated theoretical isotopic abundance of a MIRM transition of the SIL analyte or the naturally occurring isotopologues of the SIL analyte.

9. The method of any one of claims 1 to 8, wherein the analyte is a protein or a peptide.

10. The method of any one of claims 1 to 9, wherein the SIL analyte is a stable isotopically labeled protein or peptide.

11. The method of claim 10, wherein a parent ion of the SIL analyte comprises at least about 3 amino acids, at least about 4 amino acids, at least about 5 amino acids, at least about 6 amino acids, at least about 7 amino acids, at least about 8 amino acids, at least about 9 amino acids, at least about 10 amino acids, at least about 11 amino acids, at least about 12 amino acids, at least about 13 amino acids, at least about 14 amino acids, at least about 15 amino acids, at least about 16 amino acids, at least about 17 amino acids, at least about 18 amino acids, at least about 19 amino acids, or at least about 20 amino acids.

12. The method of claim 10 or 11, wherein a parent ion of the SIL analyte comprises an amino acid sequence between 4 and 20 amino acids, between 4 and 15 amino acids, between 5 and 15 amino acids, between 4 and 14 amino acids, between 5 and 14 amino acids, between 5 and 13 amino acids, between 5 and 12 amino acids, between 6 and 15 amino acids, between 6 and 14 amino acids, between 6 and 13 amino acids, between 6 and 12 amino acids, between 6 and 11 amino acids, between 6 and 10 amino acids, between 6 and 9 amino acids, between 6 and 8 amino acids, between 7 and 15 amino acids, between 7 and 14 amino acids, between 7 and 13 amino acids, between 7 and 12 amino acids, between 7 and 11 amino acids, between 7 and 10 amino acids, or between 7 and 9 amino acids.

13. The method of any one of claims 1 to 12, wherein the analyte is an antibody.

14. The method of any one of claims 1 to 12, wherein the analyte is a fusion protein.

15. The method of any one of claims 1 to 12, wherein the analyte is PD-1, PD-L1, CD73, an anti-PD-1 antibody, an anti-PD-L1 antibody, an anti-CD73 antibody, or any combination thereof.

16. The method of any one of claims 1 to 8, wherein the analyte is a small molecule.

17. The method of claim 16, wherein the small molecule has a molar mass of at least about 100 g/mol, at least about 200 g/mol, at least about 300 g/mol, at least about 400 g/mol, at least about 500 g/mol, at least about 600 g/mol, at least about 700 g/mol, at least about 800 g/mol, at least about 900 g/mol, at least about 1000 g/mol, at least about 1100 g/mol, at least about 1200 g/mol, at least about 1300 g/mol, at least about 1400 g/mol, at least about 1500 g/mol, at least about 1600 g/mol, at least about 1700 g/mol, at least about 1800 g/mol, at least about 1900 g/mol, or at least about 2000 g/mol.

18. The method of any one of claims 1 to 17, wherein the SIL analyte contains at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, or at least about 20 stable isotope labels.

19. The method of claim 18, wherein the SIL analyte contains from about 3 to about 20 isotope labels, from about 3 to about 19 isotope labels, from about 3 to about 15 isotope labels, from about 3 to about 10 isotope labels, from about 3 to about 8 isotope labels, from about 3 to about 7 isotope labels, from about 3 to about 6 isotope labels, from about 4 to about 15 isotope labels, from about 4 to about 10 isotope labels, from about 4 to about 8 isotope labels, from about 4 to about 7 isotope labels, from about 4 to about 6 isotope labels, from about 5 to about 8 isotope labels, from about 5 to about 7 isotope labels, from about 6 to about 10 isotope labels, from about 6 to about 8 isotope labels, from about 7 to about 16 isotope labels, from about 7 to about 16 isotope labels, from about 8 to about 16 isotope labels, from about 8 to about 15 isotope labels, from about 9 to about 15 isotope labels, from about 9 to about 14 isotope labels, from about 10 to about 14 isotope labels, from about 10 to about 13 isotope labels, or from about 11 to about 13 isotope labels.

20. The method of any one of claims 2 to 19, wherein each of the measured relative peak area in MIRM transitions has less than 15% deviation from the calculated theoretical isotopic abundance in the corresponding MIRM transition of the SIL analyte or the naturally occurring isotopologues of the SIL analyte.

21. The method of claim 20, wherein at least one of the measured relative peak area in MIRM transitions has less than 14%, less than 13%, less than 12%, less than 11%, less than 10%, less than 9%, less than 8%, less than 7%, less than 6%, less than 5%, less than 4%, less than 3%, less than 2%, less than 1%, less than 0.1%, less than 0.01%, less than 0.001%, or less than 0.0001%, deviation from the calculated theoretical isotopic abundance in the corresponding MIRM transition of the SIL analyte or the naturally occurring isotopologues of the SIL analyte.

22. The method of claim 21, wherein at least one of the measured relative peak area in MIRM transitions has between 1% and 15% deviation from the calculated theoretical isotopic abundance in the corresponding transition of the SIL analyte or the naturally occurring isotopologues of the SIL analyte.

23. The method of claim 22, wherein the number of the MIRM transitions is at least two, at least three, at least four, at least five, at least six, at least seven, at least 8, at least 9, at least 10, at least 11, at least 12, at least 13, at least 14, at least 15, at least 16, at least 17, at least 18, at least 19, or at least 20.

24. The method of claim 23, wherein the number of the MIRM transitions is between 2 and 20.

25. The method of any one of claims 2 to 24, wherein the analyte concentration equivalents of the highest MIRM and the lowest MIRM is at least about 10, at least about 100, at least about 200, at least about 300, at least about 400, at least about 500, at least about 600, at least about 700, at least about 800, at least about 900, at least about 1000, at least about 1100, at least about 1200, at least about 1300, at least about 1400, at least about 1500, at least about 1600, at least about 1700, at least about 1800, at least about 1900, or at least about 2000 fold difference.

26. The method of any one of claims 4 to 25, wherein the calculated theoretical isotopic abundance of two selected MIRM transitions are at least 0.01% apart, at least 0.05% apart, at least 0.1% apart, at least 0.5% apart, at least 1% apart, at least 1.5% apart, at least 2% apart, at least 2.5% apart, at least 3% apart, at least 3.5% apart, at least 4% apart, at least 4.5% apart, at least 5% apart, at least 5.5% apart, at least 6% apart, at least 6.5% apart, at least 7% apart, at least 7.5% apart, at least 8% apart, at least 8.5% apart, at least 9% apart, at least 9.5% apart, at least 10% apart, at least 20% apart, at least 30% apart, at least 40% apart or at least about 50% apart.

27. The method of any one of claims 1 to 26, wherein the SIL analyte contains less than 5%, less than 4%, less than 3%, less than 2%, less than 1%, less than 0.5%, less than 0.4%, less than 0.3%, less than 0.2%, or less than 0.1% non-labeled analyte.

28. The method of any one claims 1 to 27, wherein the label is ²H, ¹³C, ¹⁵N, ³³S, ³⁴S, ³⁶S, ¹⁷O, or ¹⁸O.

29. The method of any one of claims 1 to 28, wherein the one or more protonated or deprotonated molecular ions are singly charged, doubly charged, triply charged or higher.

30. The method of any one of claims 1 to 29, wherein the mass spectrometer is a triple quadrupole mass spectrometer comprising Q1, Q2 and Q3.

31. The method of claim 30, wherein the resolutions used for Q1 and Q3 are unit resolution.

32. The method of claim 30, wherein the resolutions used for Q1 and Q3 are different.

33. The method of claim 30, wherein the resolution used for Q1 is higher than the unit resolution of Q3.

34. The method of any one of claims 1 to 33, wherein an In-Sample Calibration Curve (ISCC) composition is added before or during the sample preparation.

35. The method of any one of claims 1 to 34, which reduces a total instrument run time.

36. The method of any one of claims 1 to 35 wherein an external calibration curve is not used.

37. The method of any one of claims 1 to 36, wherein the analyte is a biomarker.

38. The method of any one of claims 1 to 36, wherein the analyte is a metabolite.

39. The method of any one of claims 1 to 36, wherein the sample is serum, tissue, biopsy tissue, formalin fixed paraffin embedded (FFPE), plasma, saliva, cerebral spinal fluid, tear, urine, synovial fluid, dried blood spot or any combination thereof.

40. The method of any one of claims 1 to 12 and 18 to 39, wherein the analyte is CD73 or a portion thereof.

41. The method of claim 40 wherein the SIL analyte is a SIL peptide, which is V[Ile(¹³C₆, ¹⁵N)]YPAVEGR (SEQ ID NO: 1).

42. The method of any one of claims 1 to 12 and 18 to 39, wherein the analyte is PD-1 or a portion thereof.

43. The method of claim 42 wherein the SIL analyte is a SIL peptide, which is LAAFPED[Arg(¹³C₆, ¹⁵N₄)] (SEQ ID NO: 2).

44. The method of any one of claims 1 to 12 and 18 to 39, wherein the analyte is PD-L1 or a portion thereof.

45. The method of claim 44 wherein the SIL analyte is a peptide, which is LQDAG[Val(¹³C₅, ¹⁵N)]YR (SEQ ID NO: 3).

46. The method of any one of claims 1 to 8 and 16 to 39, wherein the analyte is daclatasvir.

47. The method of claim 46 wherein the SIL analyte is ¹³C₂¹⁵N₄-daclatasvir.

48. A liquid chromatography-mass spectrometry system comprising:
a liquid chromatography including at least one liquid chromatography column capable of separating an analyte from a biological matrix;
a sample comprising the analyte of interest;
at least one stable isotopically labeled analyte added to the sample; and
a mass spectrometer capable of ionizing, fragmenting, and detecting one or more protonated or deprotonated parent ions and daughter ions specific to the analyte and the stable isotopically labeled analyte.

49. A composition comprising an In-Sample Calibration Curve (ISCC) wherein ISCC comprises a stable isotopically labeled analyte.

50. A method of quantitative LC-MS/MS bioanalysis by using one-sample multipoint external calibration curve, comprising adding one or more known amount(s) of one or more analyte(s) to a blank matrix sample to construct one or more One-Sample Multipoint External Calibration Curve(s) (OSMECC) by Multiple Isotopologue Reaction Monitoring (MIRM) of each added analyte(s), wherein the MIRM of an analyte refers to multiple reaction monitoring of multiple isotope transitions of the analyte; wherein the OSMECC for each analyte is constructed in the blank matrix sample based on the relationship between the calculated theoretical isotopic abundances (analyte concentration equivalents) in the MIRM transitions and the measured tandem mass spectrometry (MS/MS) peak areas (or peak area ratios if an internal standard is used for the assay) in the corresponding MIRM transitions; wherein the concentration of the at least one analyte in a study sample is quantified using the established OSMECC in the blank matrix sample and the measured peak areas (or peak area ratios if an internal standard is used for the assay) for the analyte in the study sample from a liquid chromatography-tandem mass spectrometry (LC-MS/MS) process, wherein the peak area ratio for the analyte is the peak area of the analyte divided by the peak area of the internal standard, and wherein a tandem mass spectrometer is operated in multiple reaction monitoring mode.

51. The method of claim 50, wherein the analyte concentration equivalent for each MIRM transition is calculated from a theoretical isotopic abundance of the corresponding MIRM transition of the analyte or the naturally occurring isotopologues of the analyte, wherein the theoretical isotopic abundance is calculated using a methodology published on Analytical Chemistry, 2012, 84(11), 4844-4850, wherein the methodology is calculated based on the isotope distributions of the neutral loss and the daughter ion of the analyte.

52. The method of claim 51, wherein the theoretical isotopic abundance for each of the MIRM transition (m/z) from (p+Zₚ+α)/Zₚ to (d+Z_{d}+β)/Z_{d} of the analyte and the naturally occurring isotopologues of the analyte is calculated based on formula (I):
Isotopic abundance in an MIRM transition of (p+Zₚ+α)/Zₚ→(d+Z_{d}+β)/Z_{d}=[relative isotope distribution of the daughter ion at mass of (d+Z_{d}+β)]*[relative isotope distribution of the neutral loss at mass of n+(α−β)] (I)
Wherein m/z is the mass to charge ratio
p is the monoisotopic mass of the parent molecule of the analyte
Zₚ is the number of charge for the parent ion
d is the monoisotopic mass of the daughter fragment of the analyte
Z_{d} is the number of charge for the daughter ion
n is the monoisotopic mass of the neutral loss of the analyte
p=d+n
α and β are integer, they are the number of additional neutrons on the parent ion and daughter ion, respectively, α≥0, β≥0 and α≥β
Zₚ and Z_{d} are integers

53. The method of claim 51 or 52, wherein the isotope distribution calculator is at worldwideweb.sisweb.com/mstools/isotope.html (accessed Nov. 10, 2019).

54. The method of claim 53, wherein the highest analyte concentration equivalent (“Upper Limit of Quantification” or “ULOQ” of the ISCC) is calculated based on formula (II):
(M/V)*ng/mL (IV)
Wherein M (ng) is the total amount of the analyte added into the sample;
V is the sample volume (mL) before the analyte is added;

55. The method of claim 54, wherein one or more of the other analyte concentration equivalents in the MIRM transitions are calculated based on formula (III):
Iₐ*ULOQ (ng/ml) (III)
Wherein Iₐ is the calculated theoretical isotopic abundance of a MIRM transition of the analyte or the naturally occurring isotopologues of the analyte.

56. The method of any one of claims 50 to 55, wherein the analyte is a protein or a peptide.

57. The method of claim 56, wherein a parent ion of the analyte comprises at least about 3 amino acids, at least about 4 amino acids, at least about 5 amino acids, at least about 6 amino acids, at least about 7 amino acids, at least about 8 amino acids, at least about 9 amino acids, at least about 10 amino acids, at least about 11 amino acids, at least about 12 amino acids, at least about 13 amino acids, at least about 14 amino acids, at least about 15 amino acids, at least about 16 amino acids, at least about 17 amino acids, at least about 18 amino acids, at least about 19 amino acids, or at least about 20 amino acids.

58. The method of claim 56 or 57, wherein a parent ion of the analyte comprises an amino acid sequence between 4 and 20 amino acids, between 4 and 15 amino acids, between 5 and 15 amino acids, between 4 and 14 amino acids, between 5 and 14 amino acids, between 5 and 13 amino acids, between 5 and 12 amino acids, between 6 and 15 amino acids, between 6 and 14 amino acids, between 6 and 13 amino acids, between 6 and 12 amino acids, between 6 and 11 amino acids, between 6 and 10 amino acids, between 6 and 9 amino acids, between 6 and 8 amino acids, between 7 and 15 amino acids, between 7 and 14 amino acids, between 7 and 13 amino acids, between 7 and 12 amino acids, between 7 and 11 amino acids, between 7 and 10 amino acids, or between 7 and 9 amino acids.

59. The method of any one of claims 50 to 58, wherein the analyte is an antibody.

60. The method of any one of claims 50 to 58, wherein the analyte is a fusion protein.

61. The method of any one of claims 50 to 58, wherein the analyte is PD-1, PD-L1, CD73, an anti-PD-1 antibody, an anti-PD-L1 antibody, an anti-CD73 antibody, or any combination thereof.

62. The method of any one of claims 50 to 55, wherein the analyte is a small molecule.

63. The method of claim 62, wherein the small molecule has a molar mass of at least about 100 g/mol, at least about 200 g/mol, at least about 300 g/mol, at least about 400 g/mol, at least about 500 g/mol, at least about 600 g/mol, at least about 700 g/mol, at least about 800 g/mol, at least about 900 g/mol, at least about 1000 g/mol, at least about 1100 g/mol, at least about 1200 g/mol, at least about 1300 g/mol, at least about 1400 g/mol, at least about 1500 g/mol, at least about 1600 g/mol, at least about 1700 g/mol, at least about 1800 g/mol, at least about 1900 g/mol, or at least about 2000 g/mol.

64. The method of any one claims 51-63, wherein the number of the MIRM transitions is at least two, at least three, at least four, at least five, at least six, at least seven, at least 8, at least 9, at least 10, at least 11, at least 12, at least 13, at least 14, at least 15, at least 16, at least 17, at least 18, at least 19, or at least 20.

65. The method of claim 64, wherein the number of the MIRM transitions is between 2 and 20.

66. The method of any one of claims 51 to 65, wherein the analyte concentration equivalents of the highest MIRM and the lowest MIRM is at least about 10, at least about 100, at least about 200, at least about 300, at least about 400, at least about 500, at least about 600, at least about 700, at least about 800, at least about 900, at least about 1000, at least about 1100, at least about 1200, at least about 1300, at least about 1400, at least about 1500, at least about 1600, at least about 1700, at least about 1800, at least about 1900, or at least about 2000 fold difference.

67. The method of any one of claims 52 to 66, wherein the calculated theoretical isotopic abundance of two selected MIRM transitions are at least 0.01% apart, at least 0.05% apart, at least 0.1% apart, at least 0.5% apart, at least 1% apart, at least 1.5% apart, at least 2% apart, at least 2.5% apart, at least 3% apart, at least 3.5% apart, at least 4% apart, at least 4.5% apart, at least 5% apart, at least 5.5% apart, at least 6% apart, at least 6.5% apart, at least 7% apart, at least 7.5% apart, at least 8% apart, at least 8.5% apart, at least 9% apart, at least 9.5% apart, at least 10% apart, at least 20% apart, at least 30% apart, at least 40% apart or at least about 50% apart.

68. A method, isotope sample dilution, for quantifying a sample with the analyte concentration higher than the assay ULOQ in LC-MS/MS bioanalysis. As isotopic abundance in each MIRM channel can be calculated and measured accurately, isotope sample dilution can be achieved by simply monitoring one or a few of the MIRM channels of the analyte in addition to the most abundant MIRM channel for study samples. While the most abundant MIRM channel (isotopic abundance of 100%) is used for the quantitation of samples having concentrations within the assay calibration curve range, less abundant MIRM channels (isotopic abundance of IA %) can be used for the quantitation of samples having concentrations beyond the assay upper limit of quantitation (ULOQ), resulting in isotope dilution factors (IDF) of 100%/IA %. This approach serves as an alternate method to eliminate the need to physically dilute study samples in LC-MS/MS quantitative analysis.


SEQUENCE LISTING (3 sequences)

SEQ ID NO: 1 - protein, 9 aa, Artificial Sequence
  synthetic
VIYPAVEGR

SEQ ID NO: 2 - protein, 8 aa, Artificial Sequence
  synthetic
LAAFPEDR

SEQ ID NO: 3 - protein, 8 aa, Artificial Sequence
  synthetic
LQDAGVYR